(19) United States
(12) Patent Application Publication
(10) Pub. No.: US 2016/0075651 A1
(43) Pub. Date: Mar. 17, 2016

(54) GLYCOLIPID INHIBITION USING IMINOSUGARS

(71) Applicants: UNITHER VIROLOGY, LLC, Silver Spring, MD (US); THE CHANCELLOR, MASTERS AND SCHOLARS OF THE UNIVERSITY OF OXFORD, Oxford (GB)

(72) Inventors: Peter LAING, Cambridgeshire (GB); Raymond A. DWEK, Oxford (GB); Stephanie POLLOCK, Oxford (GB); Nicole ZITZMANN, Oxfordshire (GB); Terry BUTTERS, Oxfordshire (GB); Dominic ALONZI, Oxfordshire (GB); John KIAPPES, Oxfordshire (GB); Urban RAMSTEDT, Bethesda, MD (US)

(73) Assignees: Unither Virology, LLC, Silver Spring, MD (US); The Chancellor, Maters and Scholars of the University of Oxford, Oxford (GB)

(21) Appl. No.: 14/888,130

(22) PCT Filed: Apr. 30, 2014
(86) PCT No.: PCT/US14/36126

§ 371 Date: Oct. 30, 2015

Related U.S. Application Data
(60) Provisional application No. 61/818,621, filed on May 2, 2013.
(60) Provisional application No. 61/929,704, filed on Jan. 21, 2014.

(51) Int. Cl. C07D 211/46; A61K 31/453; A61K 31/445

(52) U.S. Cl. CPC C07D 211/46; A61K 31/445; A61K 31/453

(57) ABSTRACT

The application provides iminosugars with a high activity and specificity for inhibiting ceramide glucosyltransferase.

### CROSS-REFERENCE TO RELATED APPLICATIONS

This application claims the benefit of U.S. Provisional Application No. 61/818,621, filed May 2, 2013, and U.S. Provisional Application No. 61/929,704, filed on Jan. 21, 2014, the contents of which are hereby incorporated by reference in their entireties into the present disclosure.

### FIELD

The present application relates to iminosugars and their use as glycolipid inhibitors as well as methods of treating conditions and diseases, for which glycolipid inhibition provides a benefit.

### SUMMARY

One embodiment is a method of inhibiting ceramide glucosyltransferase and/or lowering a glycolipid concentration comprising administering to a subject in need thereof an effective amount of N-(9-Methoxynonyl)deoxynojirimycin or a pharmaceutically acceptable salt thereof.

Another embodiment is a method of inhibiting ceramide glucosyltransferase and/or lowering a glycolipid concentration comprising administering to a subject in need thereof an effective amount of a compound of Formula I or a pharmaceutically acceptable salt thereof:

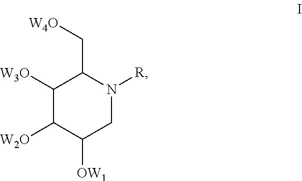

wherein R is:

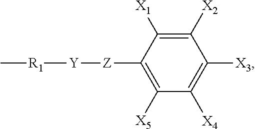

R₁ is a substituted or unsubstituted alkyl group; W₁₋₄ are independently selected from hydrogen, substituted or unsubstituted alkyl groups, substituted or unsubstituted haloalkyl groups, substituted or unsubstituted alkanoyl groups, substituted or unsubstituted aroyl groups, or substituted or unsubstituted haloalkanoyl groups; X₁₋₅ are independently selected from H, NO₂, N₃, or NH₂; Y is absent or is a substituted or unsubstituted C₁-alkyl group, other than carbonyl; and Z is selected from a bond or NH,
provided that when Z is a bond, Y is absent, and
provided that when Z is NH, Y is a substituted or substituted C₁-alkyl group, other than carbonyl.

Yet another embodiment is a method of inhibiting ceramide glucosyltransferase and/or lowering a glycolipid concentration comprising administering to a subject in need thereof an effective amount of a compound of formula II or a pharmaceutically acceptable salt thereof:

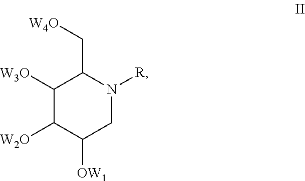

wherein R is:

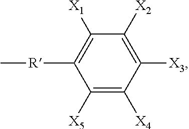

R′ is a substituted or unsubstituted alkyl group; W1-4 are independently selected from hydrogen, substituted or unsubstituted alkyl groups, substituted or unsubstituted haloalkyl groups, substituted or unsubstituted alkanoyl groups, substituted or unsubstituted aroyl groups, or substituted or unsubstituted haloalkanoyl groups; and X₁₋₅ are independently selected from H, NO₂, halogen, alkyl, or halogenated alkyl.

And yet another embodiments is a method of inhibiting ceramide glucosyltransferase and/or lowering a glycolipid concentration comprising administering to a subject in need thereof an effective amount of a compound of formula

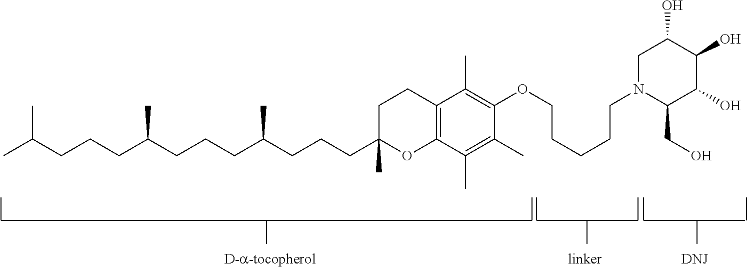 [SMILES: Cc1c(C)c2c(c(C)c1OCCCCCN1C[C@H](O)[C@@H](O)[C@H](O)[C@H]1CO)CC[C@@](C)(CCC[C@H](C)CCC[C@H](C)CCCC(C)C)O2]

or a pharmaceutically acceptable salt thereof

And yet another embodiment is a method of inhibiting glycolipid biosynthesis in cells capable of producing glycolipids comprising subjecting said cells to a glycolipid inhibitory effective amount of N-(9-Methoxynonyl)deoxynojirimycin or a pharmaceutically acceptable salt thereof

And still another embodiment is a method of inhibiting glycolipid biosynthesis in cells capable of producing glycolipids comprising subjecting said cells to a glycolipid inhibitory effective amount of a compound of Formula I or a pharmaceutically acceptable salt thereof

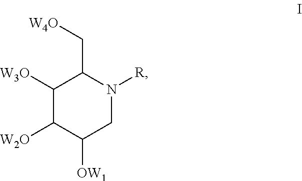

wherein R is:

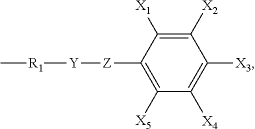

R₁ is a substituted or unsubstituted alkyl group; W₁₋₄ are independently selected from hydrogen, substituted or unsubstituted alkyl groups, substituted or unsubstituted haloalkyl groups, substituted or unsubstituted alkanoyl groups, substituted or unsubstituted aroyl groups, or substituted or unsubstituted haloalkanoyl groups; X₁₋₅ are independently selected from H, NO₂, N₃, or NH₂; Y is absent or is a substituted or unsubstituted C₁-alkyl group, other than carbonyl; and Z is selected from a bond or NH,
provided that when Z is a bond, Y is absent, and
provided that when Z is NH, Y is a substituted or substituted C₁-alkyl group, other than carbonyl.

Yet another embodiment is a method of inhibiting glycolipid biosynthesis in cells capable of producing glycolipids comprising subjecting said cells to a glycolipid inhibitory effective amount of a compound of formula II or a pharmaceutically acceptable salt thereof:

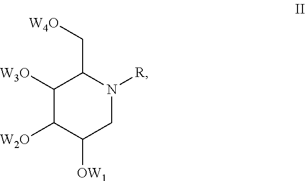

wherein R is:

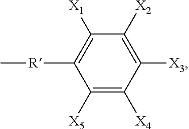

R′ is a substituted or unsubstituted alkyl group; W₁₋₄ are independently selected from hydrogen, substituted or unsubstituted alkyl groups, substituted or unsubstituted haloalkyl groups, substituted or unsubstituted alkanoyl groups, substituted or unsubstituted aroyl groups, or substituted or unsubstituted haloalkanoyl groups; and X₁₋₅ are independently selected from H, NO₂, halogen, alkyl, or halogenated alkyl.

And another embodiment is a compound of formula I

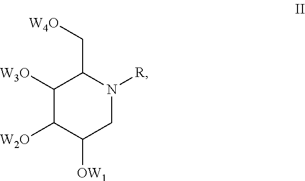

wherein R is:

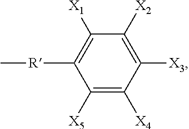

R′ is a substituted or unsubstituted alkyl group; W₁₋₄ are independently selected from hydrogen, substituted or unsubstituted alkyl groups, substituted or unsubstituted haloalkyl groups, substituted or unsubstituted alkanoyl groups, substituted or unsubstituted aroyl groups, or substituted or unsubstituted haloalkanoyl groups; and X₁₋₅ are independently selected from H, NO₂, halogen, alkyl, or halogenated alkyl.

### FIGURES

FIG. 1A-B present Hill plots of the dose response inhibition of GM3 synthesis in HL60 cells for NB-DNJ (FIG. 1A) and UV-4 (FIG. 1B). Following compound dosing of cells, GSL were extracted, the oligosaccharide cleaved and labeled with 2-AA and separated by NP-HPLC. The peak area of oligosaccharide derived from GM3 was quantified and expressed relative to untreated cells. A four parameter logistic model was used to calculate IC₅₀ values.

FIG. 2 illustrates Cellular Target of Substrate Reduction Therapy (SRT).

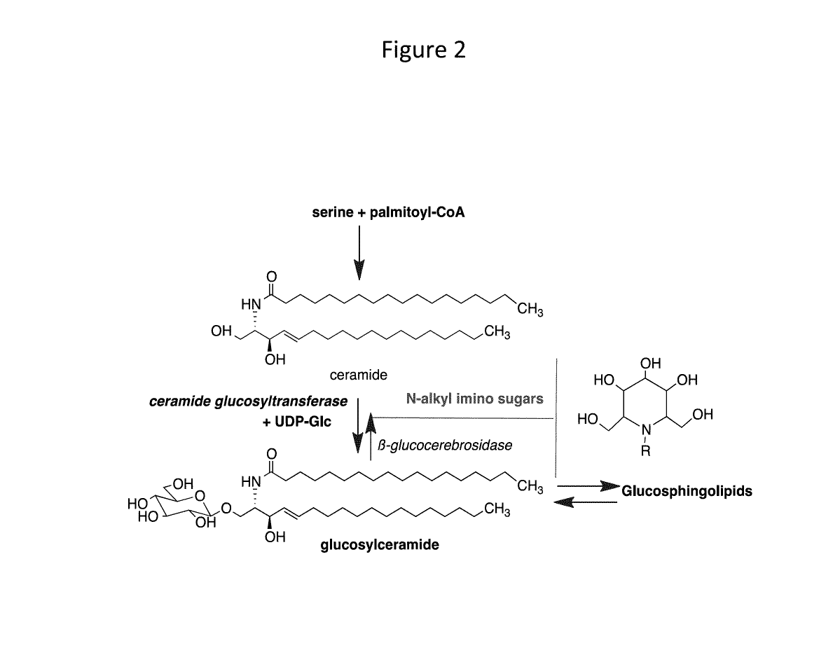

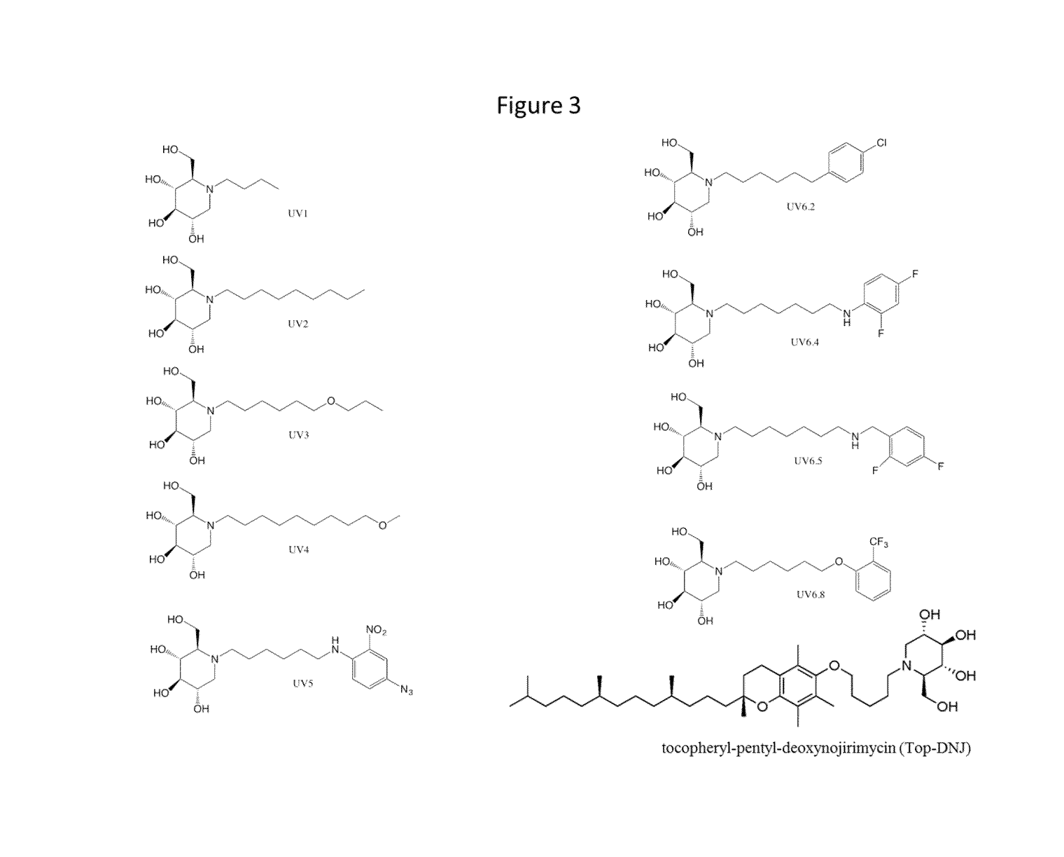

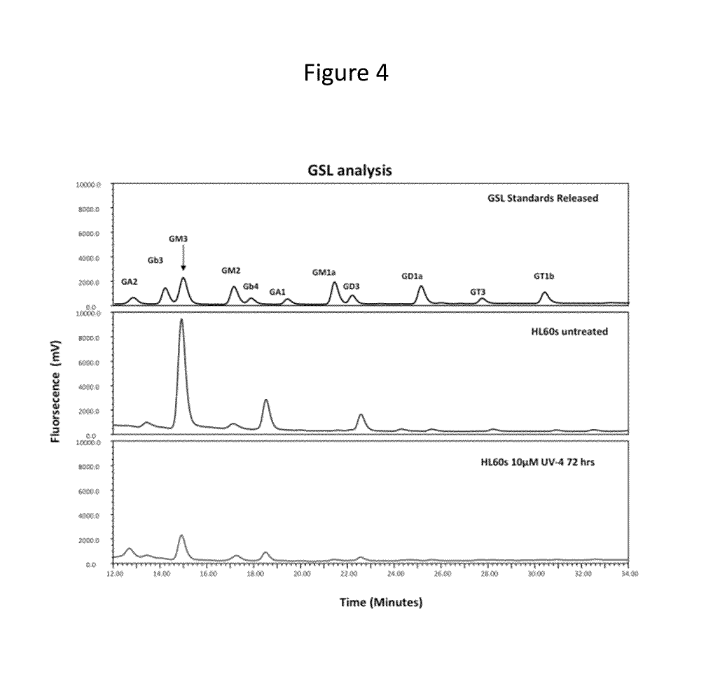

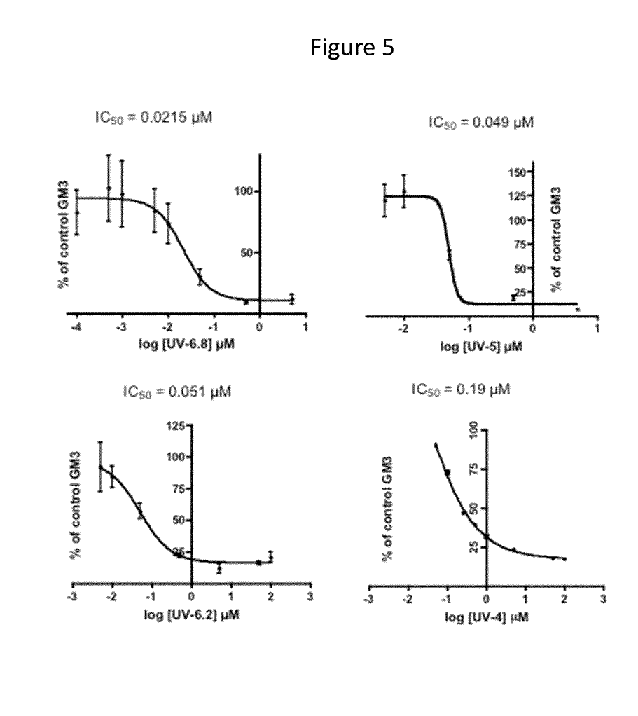

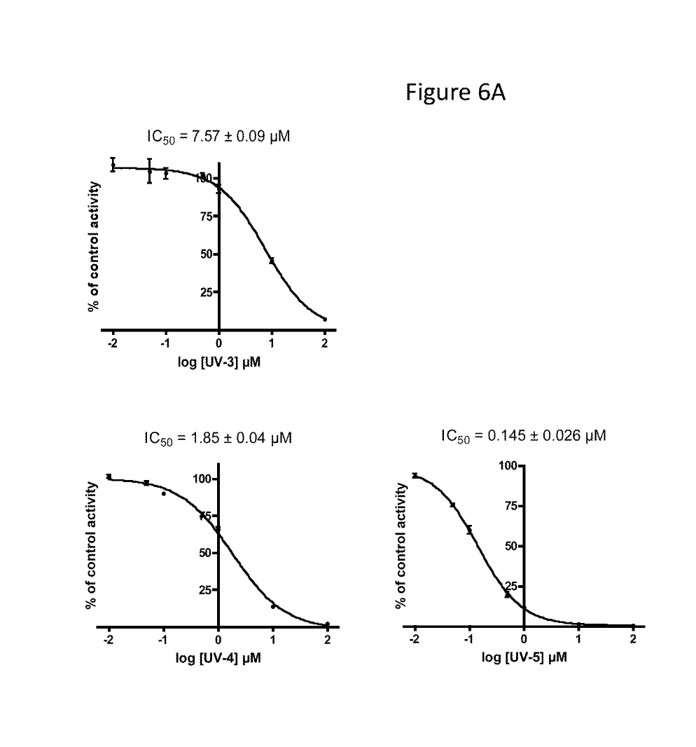

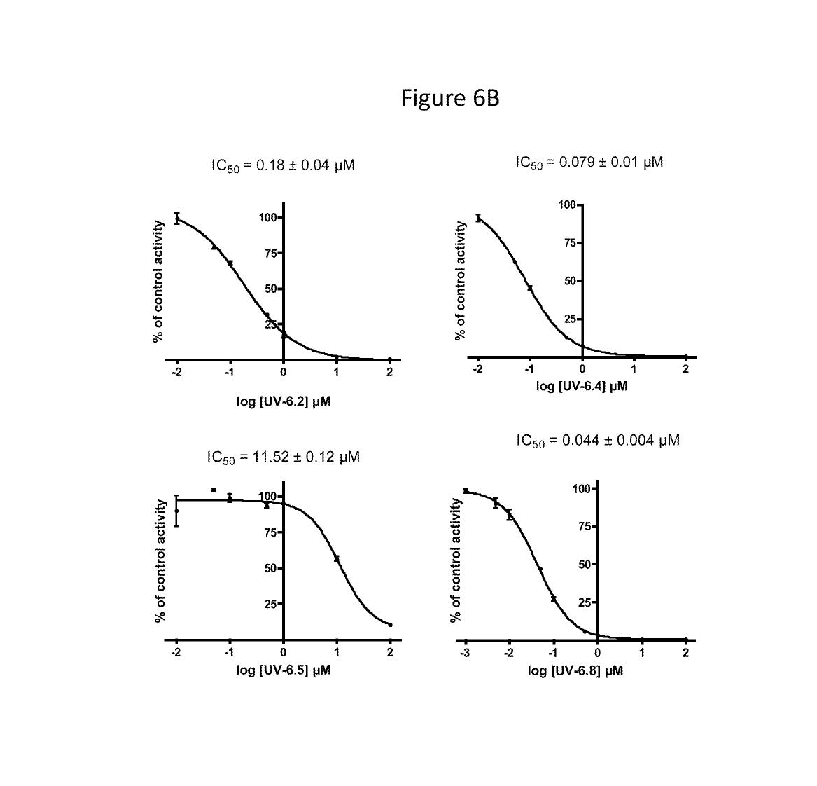

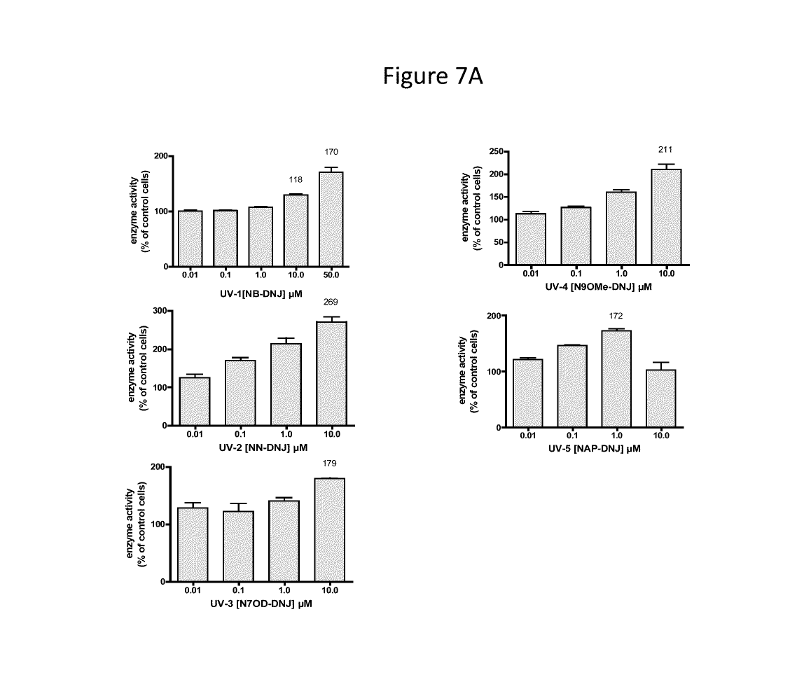

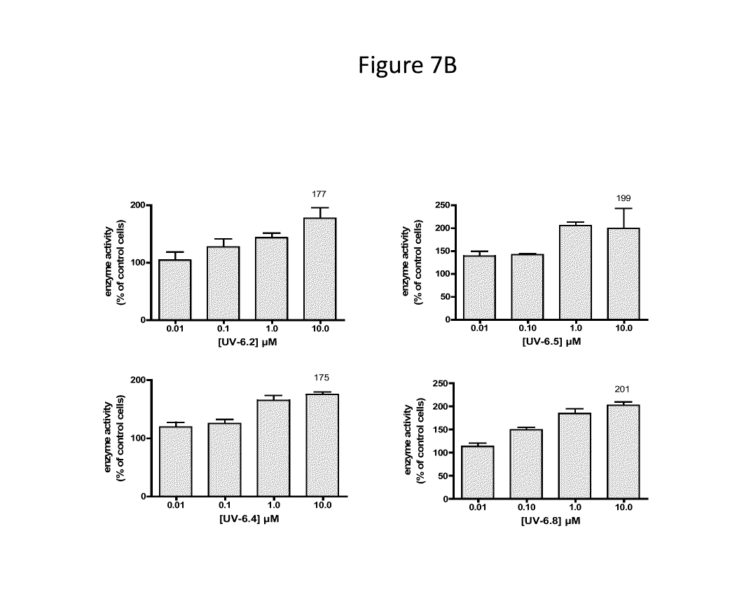

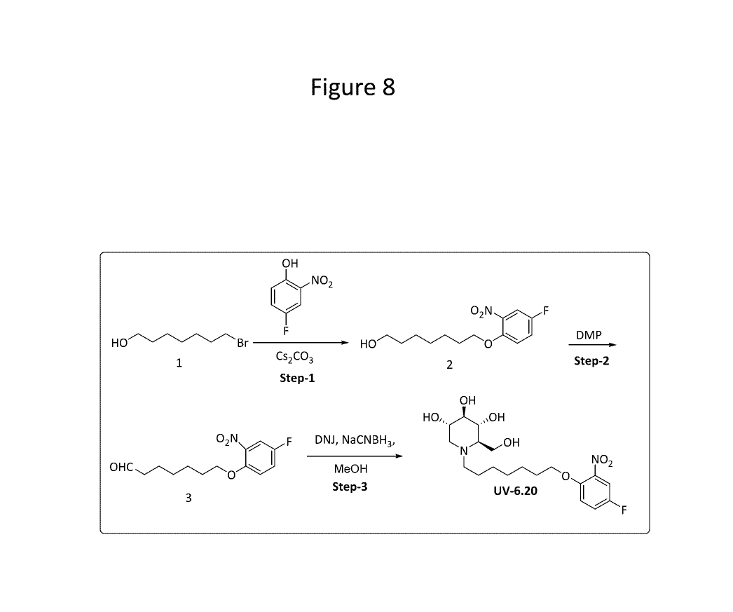

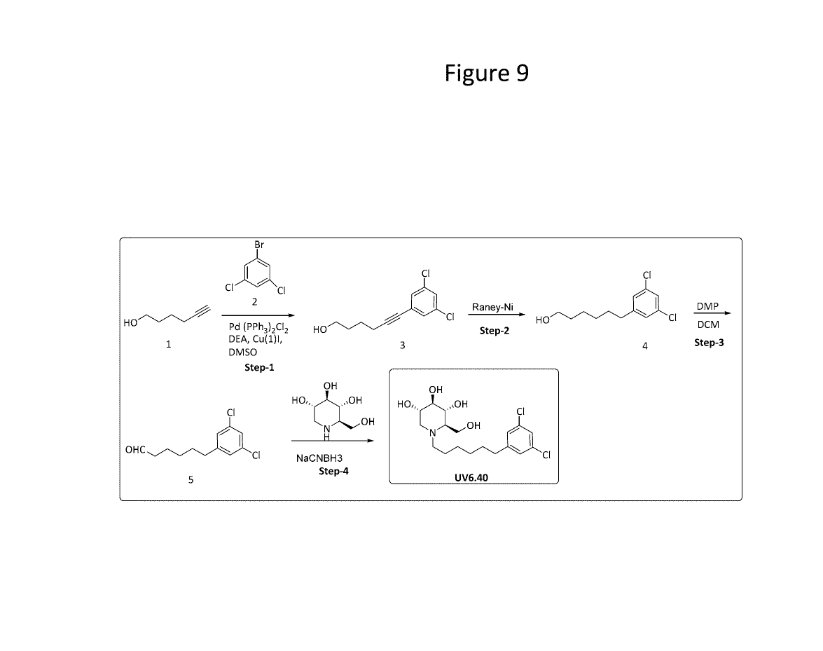

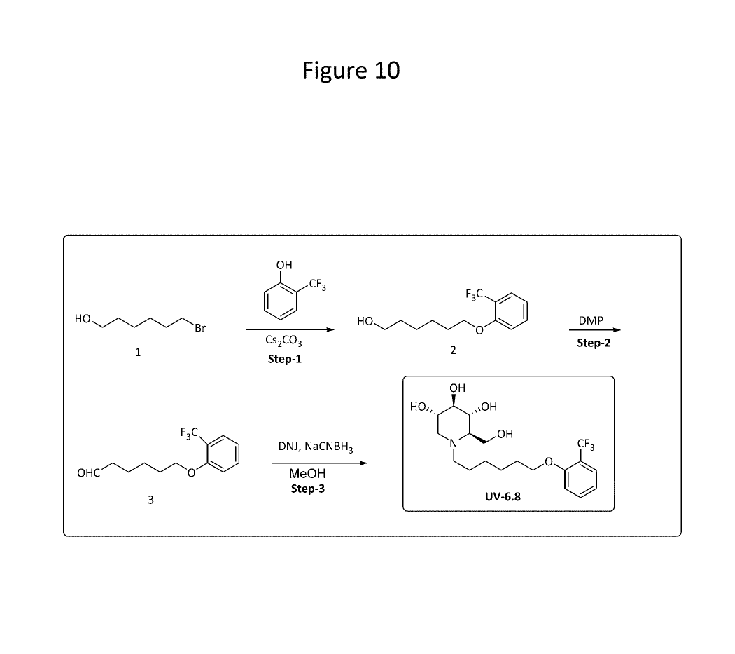

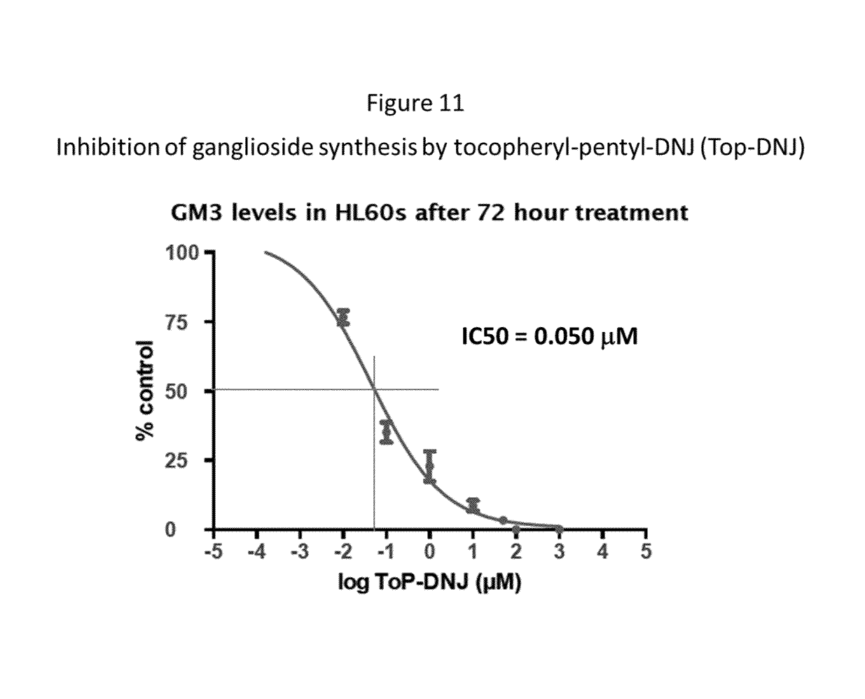

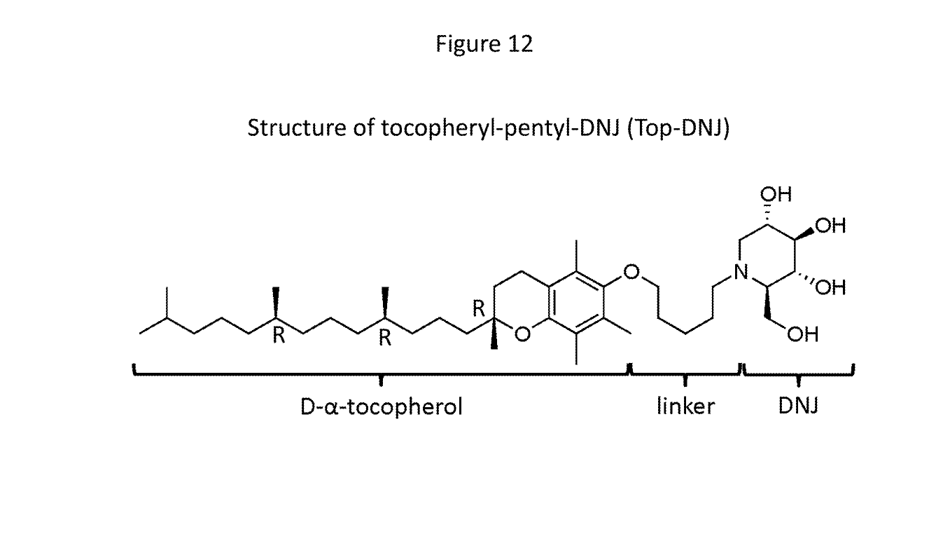

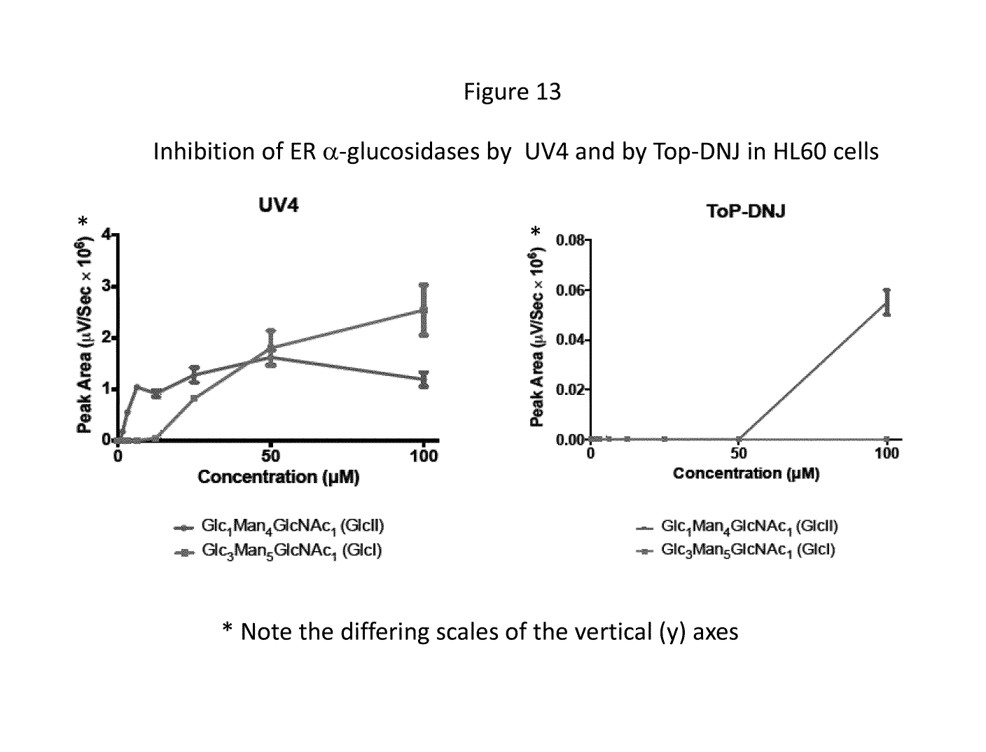

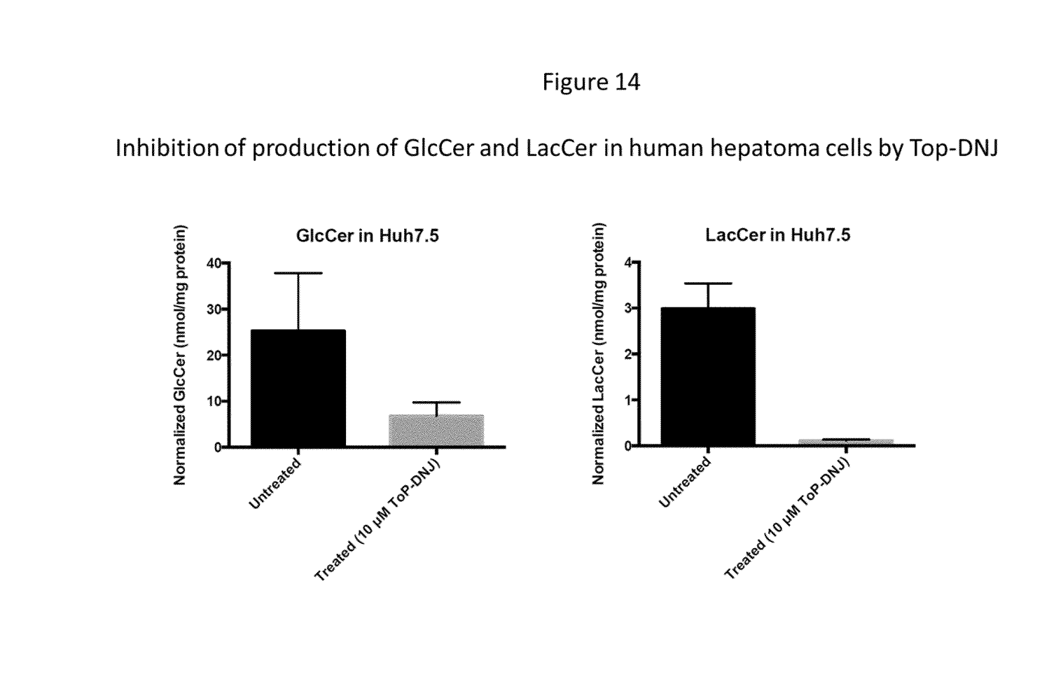

Ceramide glucosyltransferase (CGT) inhibition by N-alkylated imino sugars of gluco- and galacto-stereochemistry as treatment of Lysosomal Storage Disorders (LSD). Uridine dihosphate glucose (UDP-glucose) ceramide glucosyltransferase catalyzes the first glycosylation step in glycosphingolipid biosynthesis. The product, glucosylceramide, is the core structure of more than 300 GSLs. Although FIG. 2 mentions N-alkyl iminosugars, it should be understood that the mechanisms illustrated in this Figure may apply not only to N-alkyl iminosugars but to other N-substituted iminosugars, such the iminosugars presented in FIGS. 3 and 12.

FIG. 3 provides chemical formulae of the iminosugars used in the study.

FIG. 4 shows GSL profile of HL60 cells. HL60 cells were treated for 72 hours with varying concentrations of imino sugars. Lipids were extracted from HL60 cell pellets and characterized by labeling with 2AA and NP-HPLC analysis. GSL standard release is a positive control for the both enzyme release and fluorescent labeling. GM3 levels measured for IC₅₀ calculations

FIG. 5 shows representative Hill plots from GM3 cellular reduction assay.

Measurement of the GM3 peak area was used to determine inhibition of GSL biosynthesis. Experiments were conducted in triplicate and the error bars show standard deviations.

FIGS. 6A-B show representative Hill plots from in vitro β-glucocerebrosidase assay.

Measurement of the inhibition of human placental β-glucocerebrosidase. Experiments were conducted in triplicate and the error bars show standard deviations.

FIGS. 7A-B provide data showing enzyme enhancement upon chaperone treatment.

Gaucher N370S fibroblast treated with non-cytotoxic levels of imino sugars up to 10 μM for 3 days before harvesting and assaying of enzyme levels in comparison to untreated mutant fibroblasts.

FIG. 8 provides a synthesis scheme for making UV 6.2.

FIG. 9 provides a synthesis scheme for making UV 6.4.

FIG. 10 provides a synthesis scheme for making UV 6.8.

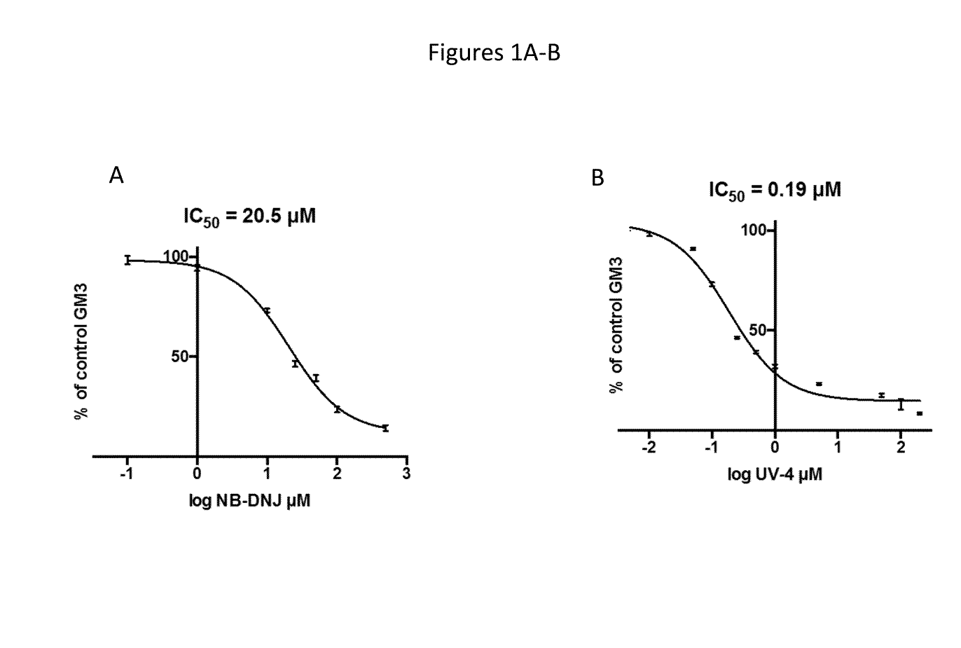

FIG. 11 presents Hill plot of the dose response inhibition of GM3 synthesis in HL60 cells for ToP-DNJ. The methodology for obtaining the data in FIG. 11 was the same as for FIGS. 1 and 5.

FIG. 12 provides chemical formula for ToP-DNJ.

FIG. 13 provides results for free oligosaccharide analysis (a measure of ER alpha glucosidase inhibition) for UV-4 and ToP-DNJ demonstrating Top-DNJ to be virtually devoid of ER glucosidase inhibitory activity. Free glucosyl oligosaccharides were measured according to Alonzi et al. Biochem. J. (2008) 409, 571-580.

FIG. 14 demonstrates the effect of Top-DNJ on cellular glucosylceramide and its downstream product lactosylceramide (an intermediate in ganglioside biosynthesis) in human hepatoma cells, exhibiting near-complete inhibition of this pathway at 10 μM concentration of Top-DNJ. Glycosphingolipids (including GlcCer and LacCer) were measured according to Wolf, C. and Quinn, P. J. Progress in lipid research (2008) 47, 15-36. Briefly, chloroform-methanol extracts of cellular lipids were subjected to HPLC to isolate the glycosphingolipid species from other cellular lipids, then subjected to two-dimentional mass-spectromertry with internal standards in order to quantify particular glycosphingolipid species.

### DETAILED DESCRIPTION

Unless otherwise specified, “a” or “an” means “one or more.”

The term “GCS” as used herein means ceramide glucosyltransferase also known as ceramide glucosyltransferase EC 2.4.1.80 or as UDP-glucose-ceramide glucosyltransferase or glucosylceramide synthase.

The term “disease” or “condition” denotes disturbances and/or anomalies that as a rule are regarded as being pathological conditions or functions, and that can manifest themselves in the form of particular signs, symptoms, and/or malfunctions.

As used herein, the terms “treat,” “treating,” “treatment,” and the like refer to eliminating, reducing, or ameliorating a disease or condition, and/or symptoms associated therewith. Although not precluded, treating a disease or condition does not require that the disease, condition, or symptoms associated therewith be completely eliminated. As used herein, the terms “treat,” “treating,” “treatment,” and the like may include “prophylactic treatment,” which refers to reducing the probability of redeveloping a disease or condition, or of a recurrence of a previously-controlled disease or condition, in a subject who does not have, but is at risk of or is susceptible to, redeveloping a disease or condition or a recurrence of the disease or condition. The term “treat” and synonyms contemplate administering a therapeutically effective amount of a compound of the invention to a subject in need of such treatment. Such a subject may be a warm-bloodied animal, such as a mammal. In many embodiments, the subject may be a human being.

The term “therapeutically effective amount” or “effective dose” as used herein refers to an amount of the active agent(s), such as an iminosugar, that is(are) sufficient, when administered by a method of the invention, to efficaciously deliver the active agent(s), such as an iminosugar, for the treatment of condition or disease of interest to an individual in need thereof. In the case of a lysosomal storage disorder, the therapeutically effective amount of the agent may reduce (i.e., retard to some extent and preferably stop) unwanted glycolipid accumulation and/or relieve, to some extent, one or more of the symptoms associated with the disorder. Preferably, the effective amount is medically beneficial but does not present toxic effects which overweigh the advantages which accompany its use.

IC50 or IC90 (inhibitory concentration 50 or 90) may be a concentration of an glycosphingolipid biosynthesis inhibiting agent, such as an iminosugar, used to achieve 50% or 90% reduction of a particular glycosphingolipid.

The present inventors discovered that certain iminosugars may be potent inhibitors of ceramide glucosyltransferase and/or have high activity at lowering the cellular concentration of glucosylceramide, lactosylceramide, and gangliosides derived from lactosylceramide. In particular, these iminosugars have a ceramide glucosyltransferase inhibiting activity and/or activity at lowering the cellular concentration of glucosylceramide, lactosylceramide, and gangliosides derived from lactosylceramide surprisingly higher than N-butyl deoxynojirimycin (NB-DNJ), which is a compound known for such activities, see e.g. U.S. Pat. Nos. 5,472,969 and 5,525,616. A number of GCS and glycosphingolipid inhibitors have been disclosed, for example, in U.S. Pat. Nos. 5,302,609; 5,472,969; 5,525,616; 5,916,911; 5,945,442; 5,952,370; 6,030,995; 6,051,598; 6,255,336; 6,569,889; 6,610,703; 6,660,794; 6,855,830; 6,916,802; 7,253,185; 7,196,205; and 7,615,573. Additional GCS inhibitors and treatments are disclosed in WO 2008/150486; WO 2009/1 17150; and WO 2010/014554.

In some embodiments, an iminosugar may be N-(9-methoxynonyl)deoxynojirimycin (UV-4) or a pharmaceutically acceptable salt thereof. N-(9-methoxynonyl)deoxynojirimycin and methods of its making are disclosed, for example, in U.S. Pat. Nos. 8,450,345 and 8,426,445 as in US patent application publications nos. 2010/0222384, 2011/0065754, 2011/0065753 and 2011/065752.

In some embodiments, an iminosugar may be a compound disclosed in US patent application publication no. 2007/0275998. For example, an iminosugar may be a compound of Formula I or a pharmaceutically acceptable salt thereof:

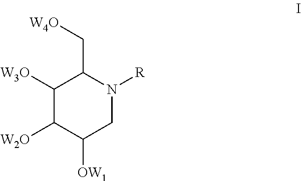

wherein R is:

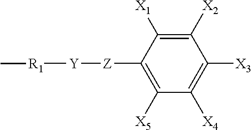

R₁ is a substituted or unsubstituted alkyl group;

W₁₋₄ are independently selected from hydrogen, substituted or unsubstituted alkyl groups, substituted or unsubstituted haloalkyl groups, substituted or unsubstituted alkanoyl groups, substituted or unsubstituted aroyl groups, or substituted or unsubstituted haloalkanoyl groups;

X₁₋₅ are independently selected from H, NO₂, N₃, or NH₂;

Y is absent or is a substituted or unsubstituted C₁-alkyl group, other than carbonyl; and

Z is selected from a bond or NH,

provided that when Z is a bond, Y is absent, and
provided that when Z is NH, Y is a substituted or substituted C₁-alkyl group, other than carbonyl. The definitions of chemical groups may be the same as US 2007/0275998.

In some embodiments, R₁ may be a substituted or unsubstituted C1-C12 alkyl group, i.e. a substituted or unsubstituted alkyl group having 1 to 12 carbons atoms. For example, R₁ may be a substituted or unsubstituted C1-C10 alkyl group or substituted or unsubstituted C3-C9 alkyl group or substituted or unsubstituted C5-C8 alkyl group. In some embodiments, D₁ may be substituted or unsubstitued butyl, pentyl, hexyl, heptyl or octyl group.

In many embodiments, Z being NH may be preferred. In such a case, Y is a substituted or substituted Cl-alkyl group, other than carbonyl.

In some embodiments, at least one or at least two of X₁₋₅ may be selected from NO₂, N₃ and NH₂. In some embodiments, at least one or at least two of X₁₋₅ may be selected from NO₂ and N₃. In some embodiments, at least one or at least two of X₁₋₅ may be selected from NO₂ and NH₂. In some embodiments, at least one or at least two of X₁₋₅ may be selected from NH₂ and N₃.

In some embodiments, the compound of Formula I may be a deoxynojirimycin derivative, i.e. a compound of Formula Ia:

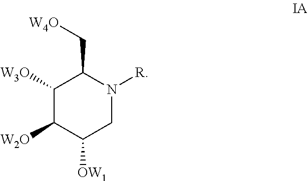

Examples of DNJ derivatives include N—(N′-{4′-azido-2′-nitrophenyl)-6-aminohexyl)-deoxynojirimycin (NAP-DNJ or UV-5) and N—(N′-{2,4-dinitrophenyl)-6-aminohexyl)-deoxynoj irimycin (NDP-DNJ).

In some embodiments, an iminosugar may be a compound of formula II or a pharmaceutically acceptable salt thereof:

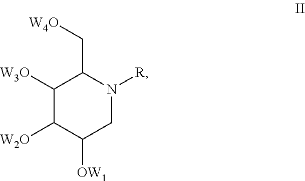

wherein R is:

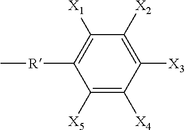

R′ is a substituted or unsubstituted alkyl group;

W₁₋₄ are independently selected from hydrogen, substituted or unsubstituted alkyl groups, substituted or unsubstituted haloalkyl groups, substituted or unsubstituted alkanoyl groups, substituted or unsubstituted aroyl groups, or substituted or unsubstituted haloalkanoyl groups; and X₁₋₅ are independently selected from H, NO₂, halogen, alkyl, or halogenated alkyl. The term substituted may have the same meaning as in US 2007/0275998. Compounds of formula II may be prepared, for example, following synthesis schemes similar to the ones depicted in FIGS. 8-10.

In some embodiments, R′ may be a substituted or unsubstituted C₁-C₁₂ alkyl group, or substituted or unsubstituted C₂-C₁₀ alkyl group or substituted or substituted C₃-C₉ alkyl group or substituted or unsubstitued C₅-C₈ alkyl group. In some embodiments, R′ may be an unsubstituted C₁-C₁₂ alkyl group, or C₂-C₁₀ alkyl group or C₃-C₉ alkyl group or C₅-C₈ alkyl group. Yet in some embodiments, R′ may be an alkyl group, such as C₁-C₁₂ or C₂-C₁₀ or C₃-C₉ or C₅-C₈ alkyl group, substituted with 1 to 3 oxygen atoms. For example, in some embodiments, R′ may be (CH₂)ₘ—O—(CH₂)ₘ, where n is 3-10 or 5-8 and m is 0-4. In some embodiments, R′ may be an amino-substituted alkyl group, i.e. an alkyl group, such as C₁-C₁₂ or C₂-C₁₀ or C₃-C₉ or C₅-C₈ alkyl group, substituted with aminogroup. For example, R′ may be (CH₂)ₚ—NH—(CH₂)_{q}, where n is 3-10 or 5-8 and q is 0-2 or 0-4.

In some embodiments, at least one or at least two of X₁₋₅ in the compound of Formula II may be halogen, such as F, Cl or Br, or halogenated alkyl. Halogenated alkyl may be C₁ halogenated alkyl, such as CHC1₂, CHF₂, CH₂C1, CH₂F, CF₃ or CCl₃.

In some embodiments, at least one of X₃ and X₅ is halogen, NO₂ or halogenated alkyl and X₁, X₂ and X₄ are H.

In some embodiments, at least one of X₃ and X₅ is F or Cl.

In many embodiments, W₁, W₂, W₃ and W₄ may be each hydrogen.

In some embodiments, the compound of Formula II may be a deoxynojirimycin derivative, i.e. a compound of formula IIa:

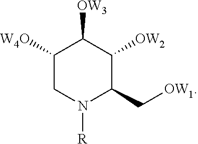

Examples of such compounds include UV-6.2, UV 6.4, UV 6.5 and UV 6.8 presented in FIG. 3.

In some embodiments, the compound of formula II or IIa may have R being one of

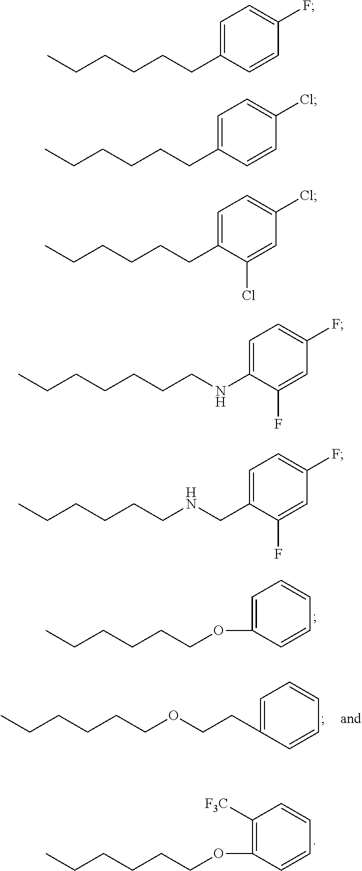

In some embodiments, an iminosugar may be a compound disclosed in US patent application publication no. 2013/0331578, which is incorporated by reference in its entirety. For example, in some embodiments, the iminosugar may be a compound having formula I′:

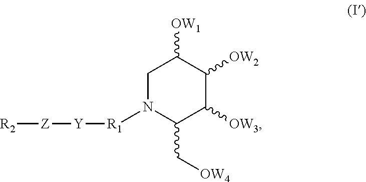

wherein:

R₁ is C₂-C₆ alkyl or oxaalkyl group;
Y is O or CH₂;

Z is selected from (CH₂)₃—O—CH₂; (CH₂)₅;

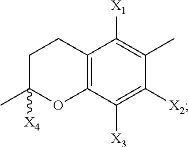

and

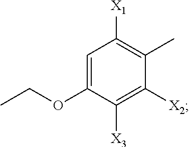

R₂ is a) straight or branched C₁₀-C₁₆ alkyl or alkylene groups and H, when Z is

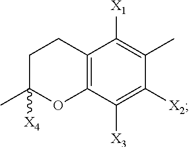

and b) straight or branched C₁₀-C₂₀ alkyl or alkylene groups, when Z is (CH₂)₃—O—CH₂; (CH₂)₅ or

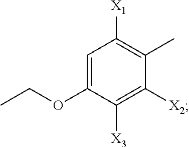

W₁₋₄ are each independently selected from H or an alcohol protecting group; and X₁₋₄ are each independently selected from H or C₁₋₂ alkyl. In some embodiments, the compound of formula I′ may be having formula II′

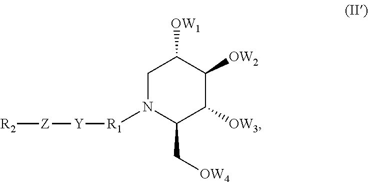

In some embodiments, R₁ may be C₅ alkyl. In some embodiments, —Z—Y— is

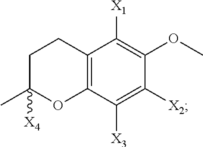

and wherein each of X₁₋₄ is independently selected from H or methyl. In some embodiments, X₄ is methyl and wherein R₂—Z—Y— is

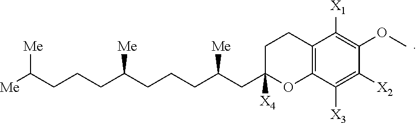

In some embodiments, X₁₋₄ are each methyl and

R₁ is C₅ alkyl. In some embodiments, W₁₋₄ are each H. In some embodiments, R₂ is

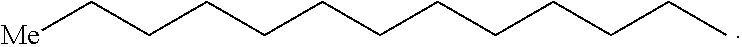

In some embodiments, the iminosugar may be a compound of the following formula:

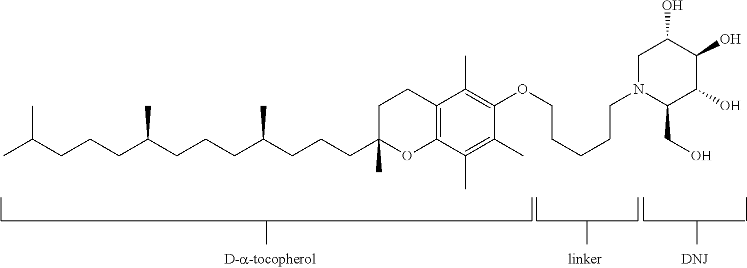 [SMILES: Cc1c(C)c2c(c(C)c1OCCCCCN1C[C@H](O)[C@@H](O)[C@H](O)[C@H]1CO)CC[C@@](C)(CCC[C@H](C)CCC[C@H](C)CCCC(C)C)O2]

(tocopheryl-pentyl deoxynojirimycin, TOP-DNJ) or a pharmaceutically acceptable salt thereof. As ceramide glucosyltransferase and/or GSL inhibitors, the above discussed iminosugars may be used for treating a number of diseases or conditions, for which inhibiting ceramide glucosyltransferase and/or lowering a glycosphingolipid concentration may be beneficial. Examples of such diseases or conditions include Gaucher disease (including Type I, Type II and Type III Gaucher disease), Fabry disease, Sandhoff disease, Tay-Sachs disease, Parkinson's disease, type II diabetes, hypertrophy or hyperplasia associated with diabetic nephropathy, an elevated blood glucose level, an elevated glycated hemoglobin level, a glomerular disease and lupus, including systemic lupus erythematosus. Examples of the glomerular disease include mesangial proliferative glomerulonephritis, collapsing glomerulopathy, proliferative lupus nephritis, crescentic glomerulonephritis and membranous nephropathy.

In some embodiments, a disease or condition, for which inhibiting ceramide glucosyltransferase and/or lowering a glycosphingolipid concentration may be beneficial, may be a lysosomal glycosphinglipid storage disease (LSD), such as Gaucher (types I, II and III) disease, Fabry disease, Sandhoff disease, Tay-Sachs disease, GM1 Gangliosidosis and Niemann-Pick Type C disease.

In some embodiments, a disease or condition, for which inhibiting ceramide glucosyltransferase and/or lowering a glycosphingolipid concentration may be beneficial, may be multiple myeloma. Many of the above disclosed iminosugars are glucosidase inhibitors in addition to being ceramide glucosyltransferase inhibitors Inhibition of osteoclastogenesis and/or reducing osteoclast activation associated with multiple myeloma with an agent, such as an iminosugar, which is a ceramide glucosyltransferase inhibitor and a glucosidase inhibitor, is disclosed in US 2011/0136868. US 2011/0136868 also discloses reducing or preventing osteolytic activity and/or bone loss with an agent, such as an iminosugar, which is a ceramide glucosyltransferase inhibitor and a glucosidase inhibitor. In some embodiments, a disease or condition, for which inhibiting ceramide glucosyltransferase and/or lowering a glycosphingolipid concentration may be beneficial, may be osteoporosis or osteoarthritis Inhibition of osteoclastogenesis and/or reducing osteoclast activation associated with these disorders will prevent bone resorption. In some embodiments, a disease or condition, for which inhibiting ceramide glucosyltransferase and/or lowering a glycosphingolipid concentration may be beneficial, may be polycystic kidney disease, including an autosomal dominant or recessive form of the polycyctic kidney disease.

In some embodiments, a disease or condition, for which inhibiting ceramide glucosyltransferase and/or lowering a glycosphingolipid concentration may be beneficial, may atherosclerosis or renal hypertrophy in a diabetic patient.

In some embodiments, a disease or condition, for which inhibiting ceramide glucosyltransferase and/or lowering a glycosphingolipid concentration may be beneficial, may be Type II diabetes and/or its related disease or condition. In some embodiments, such disease or condition may be a non-alcoholic fatty liver disease, which is a consequence of the metabolic syndrome and type II diabetes. In some embodiments, the related disease or condition may be a metabolic syndrome and/or associated dyslipidemia, which may be a precursor of type II diabetes and/or atherosclerosis. In some embodiments, the iminosugars above may be used prophylactically for the prevention of Type II diabetes and/or its related disease or condition. Although the present invention is not limited by any theory, the inventors hypothesize that the rationale for the treatment and/or prevention of Type II diabetes and/or its related disease or condition may be that an iminosugar that reduces the concentration of glucosylceramide also reduces the expression of gangliosides, especially GM3, which may result in the engagement of insulin receptor into lipid rafts, causing receptor inactivation and internalization resulting in insulin resistance. The iminosugars above may therefore deplete cells of surface GM3 and sensitize the cells to insulin, thereby being useful in the treatment of insulin resistance, which may be central to the development of, for example, metabolic syndrome, type II diabetes, non-alcoholic liver disease and atherosclerosis.

In some embodiments, the iminosugars discussed above may be used for the treatment of a bacterial diseases caused by a toxin, which binds through or to glycosphingolipid or ganglioside. For example, cholera is caused by a toxin (cholera toxin) that binds via its B-subunit to ganglioside GM1. By oral iminosugar treatment of a cholera pateint, or by colonic irrigation with an iminosugar, the expression of the GM1 target by susceptible cells in the gut epithelium may be abolished or substantially reduced, having a corresponding therapeutic effect by reducing the effect of the toxin. Another disease involving bacterial toxins is postdiarrhea hemolytic uremic syndrome, which is commonly associated with particular strains of E. coli bacteria that produce Shiga toxin type-2 which binds to the ganglioside globotriaosylceramide (Gb3). By analogy to the scenario above described for cholera therapy, the iminosugars above may be used to treat E. coli—associated disorders by reducing cellular expression of the ganglioside target of the toxin (in this case Gb3). Shiga toxin-2 is commonly expressed by E. coli 0157:H7 which is a strain of E. coli known to cause enterohemorrhagic disease. The iminosugars above may be used therefore to treat enterohemorrhagic disease associated with 0157, but also enterohemorrhagic disease caused by other bacteria that express Shiga toxin-2.

In some embodiments, for the treatment of infectious or inflammatory diseases of the gut, the nature of the headgroup of the iminosugar and of the ‘tailgroup’ may both be important. While the compounds described here may have a favorable ratio of activity against ceramide glucosyltransferase (the intended target), compared to inhibition of sucrase-isomaltase (unintended/undesirable), it may be likely that for the purpose of therapy targeting ceramide glucosyltransferase generally (and particularly for gut disorders) that iminosugar compounds lacking sucrase-isomaltase inhibitory activity would be favored. Thus, compounds disclosed in US patent application publication no. 2013/0331578, such as tocopheryl-pentyl-DNJ, may be particularly favored since (even though they have a glucose type headgroup), unlike some other DNJ-based iminosugars, they may have a very low activity against sucrase-isomaltase, while retaining high activity against ceramide glucosyltransferase. Likewise compounds having (in place of DNJ) a galactose-type or idose-type iminosugar headgroup may be particularly favored, since these headgroups may avoid inhibition of sucrase-isomaltase and the potential for dose-limiting diarrhea.

In some embodiments, the iminosugars discussed above may inhibit β-glucocerebrosidase EC 3.2.1.45 (also known as D-glucosyl-N-acylsphingosine glucohydrolase or acid beta-glucosidase). β-glucocerebrosidase is an enzyme responsible for the lysosomal catabolism of GSL including gangliosides, which is mutated in Gaucher disease giving rise to its characteristic lysosomal storage pathology. β-glucocerebrosidase is also mutated (heterozygously) in some cases of Parkinson's disease where it is a predisposing mutation found in ‘carriers’ of the Gaucher mutations. While inhibition of β-glucocerebrosidase may be, of itself, not a therapeutic objective, it so happens that compounds that are active-site directed inhibitors of this enzyme can chaperone the proper folding of certain mutant forms of the enzyme that are otherwise naturally prone to mis-fold, paradoxically increasing its catalytic activity from a the low basal levels characteristic of the Gaucher phenotype.

In some embodiments, the discussed above iminosugars may provide β-glucocerebrosidase enhancement or chaperoning to increase its activity. This property may be particularly useful, for treating Gaucher disease, particularly Type-I, but also useful for treatment of type-II and type-III Gaucher disease (i.e. the neuronopathic forms). Likewise, although the precise mechanism by which β-glucocerebrosidase mutations enhance risk of Parkinson's disease is not known, the chaperone effect of the above iminosugars might negate the pathological effect of said mutations in Parkinson's disease, by allowing proper folding of β-glucocerebrosidase and full expression of its enzymatic activity, in some cases. Furthermore, iminosugar treatment might prevent D1-dopamine receptor desensitization via caveoleae-mediated internalization, thereby enhancing the pathologically affected dopaminergic pathways in Parkinson's disease.

In some embodiments, an iminosugar may be used for treating a number of diseases or conditions, for which inhibiting GM3 synthesis and/or lowering a GM3 concentration may be beneficial. Examples of such diseases or conditions include type I Gaucher disease. In some embodiments, the discussed above iminosugars discussed above may be used for inhibiting glycolipid biosynthesis in cells (substrate reduction therapy for ganglioside storage disorders), such as mammal cells, e.g. human cells, capable of producing glycolipids by subjecting such cells to a glycolipid inhibitory effective amount of an iminosugar or its pharmaceutically acceptable salt. The term “glycolipid” as used herein includes glycolipid based molecules, such as gangliosides. In some embodiments, the glycolipids may be or may include glycosphingolipids, such as, for example, glucoceramide based glycosphingolipids. In some embodiments, the glycolipids may include one or more of gangliosides, such as GM1, GM2, GM3, GD1a, GD1b, GD2, GD3, GT1b, and GQ1. In some embodiments, the subjecting may be performed in vitro. Yet in some other embodiments, the subjecting of the cells may be performed in vivo. For example, in some embodiments, the glycolipid inhibitory effective amount or concentration of an iminosugar or its pharmaceutically acceptable salt may be administered to a subject with a disease or condition for which inhibiting glycolipid biosynthesis may be beneficial. Such a subject may be a warm blooded animal, e.g. a mammal, such as human being. Examples of such diseases or conditions include Gaucher disease (including Type I, Type II and Type III Gaucher disease), Fabry disease, Sandhoff disease, Tay-Sachs disease, GMI Gangliosidosis, Niemann-Pick Type C disease, lupus erythematosus, such as systemic lupus erythematosus, polycystic kidney disease, multiple myeloma, Giullain Barre Syndrome. The term “glycolipid inhibitory effective amount” refers to an amount or concentration of an iminosugar, which inhibits production of one or more glycolipids, without causing toxic effects which may outweigh the advantages of the iminosugar's use.

In some embodiments, an iminosugar may be in a form of a salt derived from an inorganic or organic acid. Pharmaceutically acceptable salts and methods for preparing salt forms are disclosed, for example, in Berge et al. (J. Pharm. Sci. 66:1-18, 1977). Examples of appropriate salts include but are not limited to the following salts: acetate, adipate, alginate, citrate, aspartate, benzoate, benzenesulfonate, bisulfate, butyrate, camphorate, camphorsulfonate, digluconate, cyclopentanepropionate, dodecylsulfate, ethanesulfonate, glucoheptanoate, glycerophosphate, hemisulfate, heptanoate, hexanoate, fumarate, hydrochloride, hydrobromide, hydroiodide, 2-hydroxyethanesulfonate, lactate, maleate, methanesulfonate, nicotinate, 2-naphthalenesulfonate, oxalate, palmoate, pectinate, persulfate, 3-phenylpropionate, picrate, pivalate, propionate, succinate, tartrate, thiocyanate, tosylate, mesylate, and undecanoate.

In some embodiments, an iminosugar or its pharmaceutically acceptable salt may be used as a part of a composition, which further comprises a pharmaceutically acceptable carrier and/or a component useful for delivering the composition to an animal. Numerous pharmaceutically acceptable carriers useful for delivering the compositions to a human and components useful for delivering the composition to other animals such as cattle are known in the art. Addition of such carriers and components to the composition of the invention is well within the level of ordinary skill in the art.

In some embodiments, the pharmaceutical composition may consist essentially of an iminosugar or its pharmaceutically acceptable salt, which may mean that the iminosugar or its pharmaceutically acceptable salt is the only active ingredient in the composition. In some embodiments, an iminosugar or its pharmaceutically acceptable salt may be used in a liposomal composition, such as those disclosed in US publications nos. 2008/0138351, 2009/0252785 and 2010/0266678.

Actual dosage levels of active ingredients, such as an iminosugar, in the pharmaceutical compositions may vary so as to administer an amount of the active compound(s) that is effective to achieve the desired therapeutic response for a particular patient.

The selected dose level may depend on the route of administration, the severity of the condition being treated, and the condition and prior medical history of the patient being treated. However, it is within the skill of the art to start doses of an iminosugar at levels lower than required to achieve the desired therapeutic effect and to gradually increase the dosage until the desired effect is achieved. If desired, the effective daily dose may be divided into multiple doses for purposes of administration, for example, two to four doses per day. It will be understood, however, that the specific dose level for any particular patient may depend on a variety of factors, including the body weight, general health, diet, time and route of administration and combination with other therapeutic agents and the severity of the condition or disease being treated. The adult human daily dosage may range from between about one microgram to about one gram, or from between about 10 mg and 100 mg, of iminosugar per 10 kilogram body weight. In some embodiments, a total daily dose may be from 0.1 mg/kg body weight to 100 mg/kg body weight or from 1 mg/kg body weight to 60 mg/kg body weight or from 2 mg/kg body weight to 50 mg/kg body weight or from 3 mg/kg body weight to 30 mg/kg body weight. The daily dose may be administered over one or more administering events over day. For example, in some embodiments, the daily dose may be distributed over two (BID) administering events per day, three administering events per day (TID) or four administering events (QID). In certain embodiments, a single administering event dose ranging from 1 mg/kg body weight to 10 mg/kg body weight may be administered BID or TID to a human making a total daily dose from 2 mg/kg body weight to 20 mg/kg body weight or from 3 mg/kg body weight to 30 mg/kg body weight. Of course, the amount of iminosugar which should be administered to a cell or animal may depend upon numerous factors well understood by one of skill in the art, such as the molecular weight of an iminosugar and the route of administration.

Pharmaceutical compositions that are useful in the methods of the invention may be administered systemically in oral solid formulations, ophthalmic, suppository, aerosol, topical or other similar formulations. For example, it may be in the physical form of a powder, tablet, capsule, lozenge, gel, solution, suspension, syrup, or the like. In addition to the iminosugar, such pharmaceutical compositions may contain pharmaceutically-acceptable carriers and other ingredients known to enhance and facilitate drug administration. Other possible formulations, such as nanoparticles, liposomes, resealed erythrocytes, and immunologically based systems may also be used to administer the iminosugar. Such pharmaceutical compositions may be administered by a number of routes. The term “parenteral” used herein includes subcutaneous, intravenous, intraarterial, intrathecal, and injection and infusion techniques, without limitation. By way of example, the pharmaceutical compositions may be administered orally, topically, parenterally, systemically, or by a pulmonary route.

Embodiments described herein are further illustrated by, though in no way limited to, the following working examples.

### Example 1

### Materials and Methods

### Inhibition of Glycolipid Biosynthesis

To determine the inhibition of ceramide glucosyltransferase activity in a cell-based assay, HL60 cells were cultured in the presence of various concentrations (0-500 μM) of compounds N-(9-Methoxynonyl)deoxynojirimycin (UV-4) and N-butyl-deoxynojirimycin (NB-DNJ) for 3 days until confluence, in triplicate. Cells were harvested and washed with phosphate buffered saline (PBS) before re-suspension in water and dounce homogenization. An aliquot of this homogenate was taken for protein assay. The remainder was made 4:8:3 (v/v/v) chloroform:methanol:water to extract glycolipids as described (Neville 2004, for the exact citation see section References in the end of this example). Extracted glycolipids were hydrolyzed overnight at 37° C. using a preparation of ceramide glycanase (purified in house from Hirudo medicinalis) in 20 μL of 50 mM sodium acetate buffer, pH 5.0, containing 1 mg/mL sodium taurodeoxycholate. Glycolipid-derived oligosaccharides were made to 30 μL with water and labeled with anthranilic acid (2-AA) as described below. Labeled oligosaccharides were analysed by NP-HPLC as described below (Neville 2004, Neville 2009).

### Carbohydrate Fluorescent Labelling

Glycolipid derived oligosaccharides were labeled with anthranilic acid as described previously (Neville 2004). Briefly, anthranilic acid (30 mg/mL) was dissolved in a solution of sodium acetate trihydrate (4%, w/v) and boric acid (2% w/v) in methanol. This solution was added to sodium cyanoborohydride (final concentration 45 mg/mL) and mixed to give the final labeling mixture. 2-AA labeling mixture (80 μL) was added to FOS samples (30 μL water) or glycolipid-derived oligosaccharides followed by incubation at 80° C. for 1 h. The reaction was allowed to cool to room temperature, 1 mL acetonitrile/water (97:3, v/v) was added, and the mixture was vortexed. Labeled oligosaccharides were purified by chromatography through Spe-ed Amide 2 columns (Applied Separations, Allentown, USA).

The columns were pre-equilibrated with 2×1 mL acetonitrile, 2×1 mL water followed by 2×1 mL acetonitrile. The samples were loaded using gravity flow and allowed to drip through the column. The column was washed with 2×1 mL acetonitrile/water (95:5, v/v) and labeled oligosaccharides eluted with 2×0.75 mL water.

### Carbohydrate Analysis by Normal-Phase High Performance Liquid Chromatography (NP-HPLC)

Fluorescently labeled glycolipid derived oligosaccharides were separated by NP-HPLC using a 4.6×250 mM TSKgel® Amide-80 column (Sigma, UK) according to previously published methods (Alonzi 2008, Neville 2004, 2009). The chromatography system included a Waters Alliance 2695 separations module and an in-line Waters 474 fluorescence detector set at Exλ 360 nm and Emλ, 425 nm. All chromatography was performed at 30° C. Solvent A was acetonitrile. Solvent B was Milli-Q water. Solvent C was composed of 100 mM ammonium hydroxide, titrated to pH 3.85 with acetic acid, in Milli-Q water and was prepared using a standard 5.0 N ammonium hydroxide solution (Sigma, UK). Gradient conditions were as follows: time=0 min (t=0), 71.6% A, 8.4% B, 20% C (0.8 mL mM-1); t=6, 71.6% A, 8.4% B, 20% C (0.8 mL min-1); t=6, 71.6% A, 8.4% B, 20% C (0.8 mL min-1); t=40, 52% A, 28% B, 20% C (0.8 mL min-1); t=41, 23% A, 57% B, 20% C (1.0 mL min-1); t=43, 23% A, 57% B, 20% C (1.0 mL min-1); t=44, 71.6% A, 8.4% B, 20% C (1.2 mL min-1); t=59, 71.6% A, 8.4% B, 20% C (1.2 mL mM-1); t=60, 71.6% A, 8.4% B, 20% C (0.8 mL mM-1). Samples (<50 μL) were injected in Milli-Q water/acetonitrile (1:1, v/v).

For analysis of GSL inhibition, peak areas corresponding to monosialyl-ganglioside GM3 were measured in response to inhibitor treatment to generate inhibition constants (Li et al., 2008). Inhibition constants (IC₅₀) were calculated using a four parameter logistic fit (Hill Plot, Prism software).

### Results

### Ceramide Glucosyltransferase Inhibition

To evaluate the cellular inhibition of ceramide glucosyltransferase, a key enzyme in the biosynthesis of glycosphingolipids (Butters 2000), compounds were administered to HL60 cells for 3 days. Following lipid extraction, enzymatic release of the oligosaccharide head group and fluorescence labeling, normal phase HPLC was used to analyze the effects of inhibition on biosynthesis. HL60 cells have a simple repertoire of glycolipids and the dominant species is a mono-sialylated ganglioside, GM3 (Mellor 2004) Inhibition of ceramide glucosyltransferase by imino sugars UV-4 and NB-DNJ results in the decrease in GM3 which was measured following HPLC separation. The amount of GM3 reduction as result of inhibition was analyzed to obtain IC₅₀ values (see FIG. 1). The imino sugar UV-4 was approximately 100 times more potent in cells than NB-DNJ (Zavesca), a known GSL inhibitor used for correcting GSL storage by reducing biosynthesis, in Gaucher patients.

### REFERENCES

Neville, D. C. A., et al. (2009) J Proteome Res 8, 681-687
Alonzi, D. S., et al. (2008) Biochem J 409, 571-580
Mellor, H. R., et al. (2004) Biochem J 381, 861-866
Neville, D. C. A., et al. (2004) Anal Biochem 331, 275-282
Butters, T. D., et al. (2000) Tetrahedron: Asymmetry 11, 113-124
Li, H., et al. (2008) Chem Bio Chem 9, 253-260

### Example 2

### Inhibitors of Ceramide Glucosyl Transferase and Chaperones of β-Glucosidase

A number of iminosugars based around a DNJ head group have shown a surprisingly improved efficacy on the approved drug Zavesca™ (N-butyl deoxynojirimycin, NB-DNJ) against the cellular target of ceramide glucosyltransferase. This may provide a therapeutic application for these iminosugars via reduction of glycosphingolipid (GSL) depletion. This may, for example, reduce viral receptor binding as an antiviral mechanism; provide a substrate reduction therapy (SRT illustrated in FIG. 2) against a host of glycolipid lysosomal storage disorders (LSD), such as Gaucher disease, for which Zavesca is a recognized treatment, as well as treatment of the autoimmune disease Systemic Lupus Erythematosus (Lupus) by the depletion of GSLs at the cell surface. These iminosugars may be also inhibitors, in many cases in a sub-micromolar range, of the human β-glucocerebrosidase allowing for as second therapeutic mechanism as a chaperone of the mutant enzyme, which would normally be degraded by an Endoplasmic Reticulum Associated Degradation pathway.

Lysosomal degradation of GSLs is catalyzed by glycosidases and a number of inherited diseases are seen in man where the lack of lysosomal enzyme activity, due to mutations in the gene encoding the lysosomal enzymes results in storage of the GSL in the lysosome (Butters et al, 2000a; Vellodi, 2005, for these and other citations, see References section below). Of the 40+ lysosomal storage disorder over 10 are due to sphingolipid degradation defects, for example Gaucher, Fabry, Tay-Sachs, Sandhoff disease, GM1 gangliosidosis. (Futerman & van Meer, 2004; Meikle et al, 1999) SRT is a pharmological intervention for LSD and is an alternative to enzyme replacement therapy (ERT) (Lachmann, 2010). The therapeutic strategy of SRT is to reduce GSL substrate influx by partial biosynthetic inhibition. This is a result of inhibition of ceramide glucosyltransferase (CGT) and allows the mutant catabolic enzymes in the lysosome to clear the storage burden, eventually leading to clearance.

The chemical properties for effective inhibition may be determined by in vitro assay and cellular studies (Butters et al, 2000b; Platt et al, 1994a; Platt et al, 1994b). Cellular studies may provide the greatest indication of efficacy as they allow the compounds inhibitory potential to be elucidated by taking into account both cytotoxicity but retention and cellular availability in a context that the enzyme is acting in the cell. Hence the present study demonstrates in a cellular assay improved efficacy against the CGT for a number of imninosugars described below.

Chaperone mediated therapy may be a strategy that relies upon inhibitors acting as stabilizers when enzyme activity can be deficient in the lysosome because certain newly synthesized mutation-bearing proteins are unstable and prone to misfolding. These structurally defective proteins are deemed as detected by the quality control system in the endoplasmic reticulum and subsequently diverted to cellular pathways of degradation. Competitive inhibitors for some of these lysosomal enzymes can, in subinhibitory concentrations, may act as ‘chaperones’ and rescue the mutant proteins, leading to the reconstitution of their hydrolytic activity within the lysosome (Fan, 2003).

The interaction of an iminosugar with the mutant enzyme at non-inhibitory levels may occur in the ER prior to degradation by the quality control system and allows for trafficking of the mutant enzyme which retains hydrolytic activity to the lysosome where unlike the ER lumen enzyme substrate is present in large stored amounts and coupled to a low pH environment results in dissociation of the small molecule inhibitor and increased in lysosomal enzyme activity.

Compared with enzyme replacement therapy, the plausible advantages of using small molecule inhibitors/chaperones may derive from one or more of the following: the ease of oral administration, lack of immunogenicity and the possibility of delivery across the blood-brain barrier; and thus the potential to treat neurodegenerative clinical variants.

Reduction in GSL levels at the cell surface through inhibition of ceramide glucosyltransferase may also have a therapeutic role in treatment of SLE. SLE is an autoimmune disease characterized by widespread inflammation, autoantibody production, and immune complex deposition. SLE affects nearly every organ system in the body. The underlying cause of SLE is not known but abnormalities in both B and T cells are thought to contribute to the loss of self-tolerance, production of autoantibodies, and deposition of immune complexes in the kidneys and other target tissues. These abnormalities are characterized by changing the nature of cell membrane lipids including an increase in Gb3 (possibly as a result of expression of transcription factor FLI1 regulating lupus T cell activation and IL-4 production through modulation of glycosphingolipid metabolism, specifically by mediating the breakdown pathway through the control of Neuramidase (Neul) expression and/or NEU activity during early disease), that can increase activation (Richard et al, 2013). Furthermore, increased accumulation of GSLs in cell membranes of lymphocytes increases oxidative stress and the formation of reactive oxygen species both factors that influence response and contribute to increased cardiovascular risk in SLE patients (Nandagudi et al, 2013).

The following compounds (see FIGS. 3 and 12) were shown to have an improved potency against the glycosphingolipid biosynthetic pathway enzyme (ceramide glucosyl transferase, CGT) and/or as inhibitors (and subsequently chaperones) of β-glucocerebrosidase. The approved drug Zavesca (NB-DNJ/UV-1) is shown as a positive control.

### Methods

### Cell Culture

HL60 cells and Gaucher lymphoblasts (N370S) were cultured in RPMI1640 medium supplemented with 10% or 15% (v/v) foetal bovine serum, respectively, 2 mM L-glutamine, 100 U/mL penicillin and 100 mg/mL streptomycin at 37° C. and 5% CO₂.

### Inhibition of Glycolipid Biosynthesis

To determine the inhibition of ceramide glucosyltransferase activity in a cell-based assay, HL60 cells were cultured in the presence of various concentrations (0-100 mM) of compound for 3 days until confluence. Cells were harvested and washed with phosphate buffer saline (PBS) before re-suspension in water and Dounce homogenisation. An aliquot of this homogenate was taken for protein assay. The remainder was made 4:8:3 (v/v/v) chloroform:methanol:water to extract glycolipids as described (Neville et al., 2004). Extracted glycolipids were hydrolyzed overnight at 37° C. using a preparation of ceramide glycanase (purified in house from Hirudo medicinalis) in 20 mL of 50 mM sodium acetate buffer, pH 5.0, containing 1 mg mL-1 sodium taurodeoxycholate. Glycolipid-derived oligosaccharides were made to 30 mL with water and labelled with anthranilic acid (2-AA) as described below. Labelled oligosaccharides were analyzed by NP-HPLC as described below.

### Carbohydrate Fluorescent Labelling

Freen oligosaccharide (FOS) and glycolipid derived oligosaccharides were labelled with anthranilic acid as described previously (Neville et al., 2004). Briefly, anthranilic acid (30 mg mL⁻¹) was dissolved in a solution of sodium acetate trihydrate (4%, w/v) and boric acid (2% w/v) in methanol. This solution was added to sodium cyanoborohydride (final concentration 45 mg mL⁻¹) and mixed to give the final labelling mixture. 2-AA labeling mixture (80 mL) was added to FOS samples (30 mL water) or glycolipid-derived oligosaccharides followed by incubation at 80° C. for 1 h. The reaction was allowed to cool to room temperature, 1 mL acetonitrile/water (97:3, v/v) was added, and the mixture was vortexed. Labelled oligosaccharides were purified by chromatography through Speed Amide 2 columns (Applied Separations, Allentown, USA). The columns were pre-equilibrated with 2×1 mL acetonitrile, 2×1 mL water followed by 2×1 mL acetonitrile. The samples were loaded using gravity flow and allowed to drip through the column. The column was washed with 2×1 mL acetonitrile/water (95:5, v/v) and labelled oligosaccharides eluted with 2×0.75 mL water.

### Carbohydrate Analysis by Normal-Phase High Performance Liquid Chromatography (NP-HPLC)

Glycolipid-derived oligosaccharides were separated by NP-HPLC using a 4.6×250 mM TSKgel Amide-80 column (Sigma, UK) according to previously published methods. The chromatography system consisted of a Waters Alliance 2695 separations module and an in-line Waters 474 fluorescence detector set at Emλ 360 nm and Emλ 425 nm. All chromatography was performed at 30° C. Solvent A was acetonitrile. Solvent B was Milli-Q® water. Solvent C was composed of 100 mM ammonium hydroxide, titrated to pH 3.85 with acetic acid, in Milli-Q water and was prepared using a standard 5.0 N ammonium hydroxide solution (Sigma, UK). Gradient conditions were as follows: time=0 min (t=0), 71.6% A, 8.4% B, 20% C (0.8 mL min⁻¹); t=6, 71.6% A, 8.4% B, 20% C (0.8 mL min⁻¹); t=6, 71.6% A, 8.4% B, 20% C (0.8 mL min⁻¹); t=40, 52% A, 28% B, 20% C (0.8 mL min⁻¹); t=41, 23% A, 57% B, 20% C (1.0 mL min⁻¹); t=43, 23% A, 57% B, 20% C (1.0 mL min⁻¹); t=44, 71.6% A, 8.4% B, 20% C (1.2 mL min⁻¹); t=59, 71.6% A, 8.4% B, 20% C (1.2 mL min⁻¹); t=60, 71.6% A, 8.4% B 20% C (0.8 mL min⁻¹) Samples (<50 mL) were injected in Milli-Q® water/acetonitrile (1:1, v/v).

For GSL analysis, peak areas corresponding to monosialyl-ganglioside GM3 were measured in response to inhibitor treatment to generate inhibition constants.

### β-Glucocerebrosidase Inhibition Assay

Human placental β-glucocerebrosidase was isolated and purified by a modified procedure of Furbish et al, Proc. Nat. Acad. Sci. (1977) 74 (8) 3560-3. Enzyme activity was measured in 50 ml of 5 mM 4-methylumbelliferyl-β-glucoside (4-MU-b-glucoside) in 0.1 M citrate phosphate buffer, pH 5.2 containing 0.25% sodium taurocholate, 0.1% TX100 at 37° C. for 15-60 min. The reaction was stopped by the addition of 200 ml 0.5 M sodium carbonate and the fluorescence measured at ex 350 nm, em 460 nm Inhibition constants (IC₅₀) were generated for placental β-glucocerebrosidase (Kₘ for 4-MU-β-glucoside, 1.9±0.3 mM) using 0.5 mM substrate concentration. Determinations were made in triplicate. Data were fitted using Hill Slope plots (Prizm software) and symmetrical standard errors determined for each IC₅₀ value.

### β-Glucocerebrosidase Activation Assay

Gaucher lymphoblasts (N3705) were cultured in the presence of various concentrations of inhibitor (0-50 nM) for 3 days before β-glucocerebrosidase activity was measured. Cells were washed twice in phosphate buffered saline, homogenized in water using a small dounce homogeniser, centrifuged at 800 g for 5 min and the supernatant taken for protein and β-glucocerebrosidase activity. Protein concentration was determined using the BCA assay (Pierce, UK) according to manufacturer's instructions. All enzyme activation measurements were made using aliquots of homogenate and 5 mM 4-methylumbelliferyl-β-glucoside in 0.1 M citrate phosphate buffer, pH 5.2 containing 0.25% sodium taurocholate, 0.1% TX100 as described above. Bromoconduritol (500 nM-2.5 mM) was added to some enzyme activity determinations to confirm the specific hydrolysis of substrate by β-glucocerebrosidase. Enzyme activation is defined as the fold increase in enzyme activity (U/mg protein) in treated cells compared to untreated cells.

### Results

### Ceramide Glucosyltransferase Inhibition

To evaluate the cellular inhibition of ceramide glucosyltransferase, a key enzyme in the biosynthesis of glycosphingolipids, the compounds were administered at non-toxic concentrations to HL60 cells for 3 days. Following lipid extraction, enzymatic release of the oligosaccharide head group and fluorescence labelling, normal phase HPLC (NP-HPLC) was used to analyze the effects of inhibition on biosynthesis. HL60 cells have a simple repertoire of glycolipids and the dominant species is a mono-sialylated ganglioside, GM3 Inhibition of ceramide glucosyltransferase by imino sugars results in the decrease in GM3, which was measured following HPLC separation (FIG. 4). Table 1 presents ceramide glucosyltransferase cellular assay data. IC₅₀ values were calculated using Hill plots, such as the ones in FIG. 5.

TABLE 1
Ceramide glucosyltransferase cellular assay data
Concentration of iminosugars UV1-5, UV6.2 and UV 6.8
resulting in 50% inhibition of ceramide glucosyltransferase
activity in HL60 cells, in comparison with NB-DNJ (UV1).

| Compound | IC₅₀ (μM) |
|---|---|
| UV1 | 20.1 ± 2.4 |
| UV2 | 2.13 ± 0.8 |
| UV3 | 10.3 ± 1.2 |
| UV4 | 0.190 ± 0.021 |
| UV5 | 0.049 ± 0.005 |
| UV6.2 | 0.051 ± 0.003 |
| UV6.8 | 0.022 ± 0.002 |

The data in Table 1 clearly show improved activity of over 100 fold in some cases. The data are important as although in vitro data gives a good indication of activity this assay allows for any cellular differences in access and retention of compound to be taken into account. Any variation due to access may be limited due to the cellular location of CGT being freely accessible to iminosugars Iminosugars may cross the plasma membrane quickly and efficiently such that the concentration of compound in the cytosol is at equilibrium with the extracellular concentration. N-Alkylated DNJ analogues may enter the cell rapidly where they may directly interact with the ceramide glucosyltransferase on the cytosolic side of the cis Golgi.

### β-Glucocerebrosidase Inhibition

All studied compounds showed improved inhibitory potency for human placental β-glucocerebrosidase compared to NB-DNJ, as determined by a fluorogenic assay using 4-methylumbelliferyl-β-glucoside (Table 2). IC₅₀ values in Table 2 were calculated using Hill plots, such as the ones in FIG. 6.

TABLE 2
In vitro data for human placental β-glucocerebrosidase
Concentration of iminosugars UV1-5, UV6.2, UV6.4,
UV6.5 and UV6.8 resulting in 50% inhibition of β-gluco-
cerebrosidase activity, in comparison with NB-DNJ (UV1)

| Compound | IC₅₀ (μM) |
|---|---|
| UV1 | 259 ± 22 |
| UV2 | 0.66 ± 0.04 |
| UV3 | 7.57 ± 0.09 |
| UV4 | 1.85 ± 0.04 |
| UV5 | 0.15 ± 0.03 |
| UV6.2 | 0.18 ± 0.04 |
| UV6.4 | 0.079 ± 0.01 |
| UV6.5 | 11.52 ± 0.12 |
| UV6.8 | 0.044 ± 0.004 |

The in vitro data in Table 2 show that the studied compounds have a surprising higher β-glucocerebrosidase inhibitory activity compared to UV1 (Zavesca). These data suggest that the studied compounds may act as competitive inhibitors and be able to bind to mutant enzyme in the ER and stabilize the protein to such an extent that it is able to protect it from degradation.

### Ability to Chaperone β-Glucocerebrosidase

Chaperone activity of the set of compounds in mutant Gaucher lymphoblasts with the most common N370S mutation is reported in Table 3. These data show the fold increases in β-Glucocerebrosidase activity compared to untreated cells. The full dose-response relationships are described in FIG. 7.

TABLE 3
Enhancement levels of β-glucocerebrosidase in Gaucher fibroblasts

| Compound | Activation fold at 10 μM |
|---|---|
| UV1 | 1.2 |
| UV2 | 2.7 |
| UV3 | 1.8 |
| UV4 | 2.1 |
| UV5 | 1.7 (@ 1 μM) |
| UV6.2 | 1.77 |
| UV6.4 | 1.75 |
| UV6.5 | 1.99 |
| UV6.8 | 2.01 |

Once again the studied iminosugars show an surprising enhancement in efficacy compared to UV1. The 2-fold increase is significant in terms of potential treatment as with the compounds also providing SRT the increased activity in the lysosome may well be able to elevate/clear any GSL storage problem associated with the disease.

### SUMMARY

The studied iminosugars have shown a surprisingly higher efficacy against the cellular targets compared to Zavesca (UV1). Ceramide glucosyltransferase is a therapeutic target in a number of diseases as described above, such as lysosomal storage diseases (LSD), systemic lupus erytehmatosus (SLE)), but in particular in the treatment of LSD (including Gaucher disease). The second mechanism of action for treatment of Gaucher disease (second to substrate reduction therapy defined above) is the chaperone-mediated therapy of Gaucher disease with small molecules that facilitate the proper folding of mutant β-glucocerebrosidase. This second mechanism may be effective only for patients with Gaucher disease due to the misfolded mutation N370S, because iminosugars have been shown to facilitate the proper folding of this particular mutant form of β-glucocerebrosidase. More than 300 mutations in the GBA gene have been documented, three of the five most common mutations in Ashkenazi Jews—N370S, 84GG and V394L (Fares et al, 2008). Approximately one out of every 20 Ashkenazi Jews carries a copy of the N370S mutation. About one out of every 334 carries a copy of the 84GG mutation. The V394L mutation is found in about one out of every 1,112 Ashkenazi Jews. The N370S mutation is associated only with type 1 Gaucher disease, which usually lacks neurological symptoms (Elstein et al, 2001). Since the N370S mutation is amenable to chaperone therapy, it can be seen that compounds of the present invention may, in the case of the N370S variant of type-I Gaucher disease, have a dual mechanism of action mediated partly by substrate reduction (inhibition of ceramide glucosyltransferase) and partly by the chaperone effect (promotion of the folding of β-glucocerebrosidase). This mutation allows the chaperone-mediated folding of the mutant enzyme, protecting it against eradication by the ERAD transporter in the ER and further permitting the correct trafficking of the properly folded enzyme to the lysosome, its proper destination organelle. The cellular location of these two target enzymes (β-glucocerebrosidase and CGT) may be also important since CGT is found on the cytosolic face of the Golgi apparatus, which may be clearly accessible to iminosugars, whereas the ER (where the chaperoning effect is occurring) may be much less accessible to iminosugars. However, since sub-inhibitory levels of the compounds may be required to exert chaperone effect this property may be an advantageous feature of compounds of the present invention.

### REFERENCES

Butters T D, et al (2000a) 100: 4683-4696
Butters T D, et al (2003) Advances in experimental medicine and biology 535: 219-226
Butters T D, et al (2000b) Tetrahedron-Asymmetr 11: 113-124
Cox T, et al (2000) Lancet 355: 1481-1485
Elstein D, et al (2001) Lancet 358: 324-327
Fan J Q (2003) Trends in pharmacological sciences 24: 355-360
Fares F, et al (2008) Prenatal diagnosis 28: 236-241
Futerman A H, et al (2004). Nature reviews Molecular cell biology 5: 554-565
Lachmann R (2010) Biochemical Society transactions 38: 1465-1468
Martinez M A, et al. (2013) J Virol 87: 1115-1122
Meikle P J, et al. (1999). JAMA: the journal of the American Medical Association 281: 249-254
Nandagudi A, et al. (2013) Lupus 22: 1070-1076
Platt F M, et al. (1994a) J Biol Chem 269: 8362-8365
Platt F M, et al. (1994b) J Biol Chem 269: 27108-27114
Richard E M, et al. (2013) PloS one 8: e75175
Taube S, et al. (2009) J Virol 83: 4092-4101
van Giersbergen P L, et al. (2007) Journal of clinical pharmacology 47: 1277-1282
Vellodi A (2005) British journal of haematology 128: 413-431

### Example 3

### Huh7.5 Cell Culture (FIG. 14)

Huh7.5 cells were grown in DMEM supplemented with 100 U/ml penicillin, 100 ng/ml streptomycin, 2 mM L-glutamine, 1×MEM, and 10% FBS. All incubations were at 37° C./5% CO₂. The effect of iminosugar treatment on cellular lipid profiles was determined in cells after incubation for 4 days in the presence or absence of iminosugars, at which point they were harvested using trypsin/EDTA, washed 3 times in cold PBS, counted using trypan blue staining, and final cell pellets were resuspended in methanol:acetone (vol 1:1) prior to lipid profiling, A small volume of each sample was used for total protein estimation using the Bradford protein assay (Bio-Rad).

Measurement of GlcCer and LacCer (FIG. 14):

Glucosyl Ceramide (Measured and Inferred from Measurement of ‘Glycosyl Ceramide’ Since the MS Methodology does not Distinguish Glucosyl from Galactosyl Moieties) and the Explicit Measurement of Lactosylceramide (LacCer), were Conducted as Part of a Comprehensive Lipidomic Analysis of Cellular Lipids as Follows.

The methodology has been described in detail previously (Wolf, C., Quinn, P. J., Lipidomics: Practical aspects and applications. Progress in Lipid Research 2008, 47, 15-36: Quinn, P. J., Rainteau, D., Wolf, C., Lipidomics of the red cell in diagnosis of human disorders. Methods Mol Biol 2009, 579, 127-159). Pellets of cultured hepatoma Huh7.5 cells were extracted with chloroform using the method of Bligh & Dyer (Bligh, E. G., Dyer, W. J., A Rapid Method of Total Lipid Extraction and Purification Canadian Journal of Biochemistry and Physiology 1959, 37, 911-917). Chloroform extracts were subjected to HPLC (Agilent 1200 Series) on a polyvinyl-alcohol functionalized silica column (PVASil, YMC, ID 4 mm, length 250 mm, Interchim, Montluçon 03100, France) in order to separate out the various lipid classes. Less polar lipids (triglycerides, diglycerides, cholesterol esters, ceramides, glucosyl- and lactosylceramides) are eluted between 5 and 15 minutes by the solvent system hexane/isopropanol/water ammonium acetate 10 mM (40/58/2 vol/vol). Phospholipids were subsequently eluted by the solvent hexane/isopropanol/water ammonium acetate 10 mM (40/50/10 vol/vol) as a function of an increasing polarity between 15 and 60 minutes in the following order:phosphatidylethanolamine, lysophosphatidylethanolamine, phosphatidylserine, phosphatidylinositol, phosphatidylcholine, sphingomyelin, lysophosphatidylcholine. Eluted lipids were channeled into the electrospray interface of the spectrometer (Turbolon, Framingham, Mass. 01701, USA). The lipid ionization was run in positive mode for M+NH₄⁺ and M+H⁺ detection. The source was coupled to a triple quadrupole mass spectrometer (API3000, ABSciex, Toronto, Canada) run in the “collision induced dissociation” mode (or “precursor” mode) for monitoring the characteristic fragment ions of the successively eluted lipid classes. Precursor molecular species of the characteristic fragment ion were identified in a library prepared for cultured hepatoma cells with the software LIMSA (Haimi, P., Chaithanya, K., Kainu, V., Hermansson, M., Somerharju, P., Instrument-independent software tools for the analysis of MS-MS and LC-MS lipidomics data. Methods in molecular biology (Clifton, N.J.) 2009, 580). Molecular species of lipids being identified, a list of ion pairs (precursor/product ion) was prepared for quantification by multiple reaction monitoring (MRM). The corresponding MRM peaks are time-integrated. The lipid amounts were calculated relative to the appropriate lipid class standard assuming an even response coefficient of all molecular species in the class.

Statistical procedures comparing the profiles were performed using the software XLStat® (version 2011. 2; Addinsoft, France). Parametric tests, multivariate analysis, correlation tests and regression procedures were applied as detailed in (Golmard, J. L., 2012, Analyse Statistique des Donnees, Edition Ellipses, Paris 75740 Cedex 15, France).

Although the foregoing refers to particular preferred embodiments, it will be understood that the present invention is not so limited. It will occur to those of ordinary skill in the art that various modifications may be made to the disclosed embodiments and that such modifications are intended to be within the scope of the present invention.

All of the publications, patent applications and patents cited in this specification are incorporated herein by reference in their entirety.

What is claimed is:

1. A method of inhibiting ceramide glucosyltransferase and/or lowering a glycolipid concentration comprising administering to a subject in need thereof an effective amount of N-(9-Methoxynonyl)deoxynojirimycin or a pharmaceutically acceptable salt thereof.

2. A method of inhibiting ceramide glucosyltransferase and/or lowering a glycolipid concentration comprising administering to a subject in need thereof an effective amount of a compound of Formula I or a pharmaceutically acceptable salt thereof: 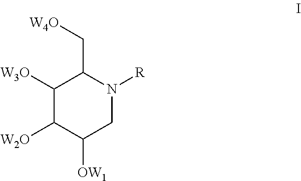
wherein R is: 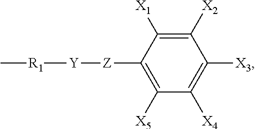
R₁ is a substituted or unsubstituted alkyl group;
W₁₋₄ are independently selected from hydrogen, substituted or unsubstituted alkyl groups, substituted or unsubstituted haloalkyl groups, substituted or unsubstituted alkanoyl groups, substituted or unsubstituted aroyl groups, or substituted or unsubstituted haloalkanoyl groups;
X₁₋₅ are independently selected from H, NO₂, N₃, or NH₂;
Y is absent or is a substituted or unsubstituted C₁-alkyl group, other than carbonyl; and
Z is selected from a bond or NH,
provided that when Z is a bond, Y is absent, and
provided that when Z is NH, Y is a substituted or substituted C₁-alkyl group, other than carbonyl.

3. The method of claim 2, wherein R₁ is a substituted or unsubstituted butyl, pentyl, hexyl, heptyl, or octyl group.

4. The method of claim 2, wherein Z is NH.

5. The method of claim 2, wherein at least one of X₁₋₅ is selected from NO₂, N₃ or NH₂.

6. The method of claim 2, wherein the compound of Formula I has the structure of the compound of Formula IA:

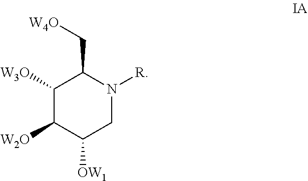

7. The method of claim 6, wherein
R₁ is —(CH₂)₅—;
W₁₋₄ are H;
X₁ is NO₂;
X₃ is N₃;
X₂, X₄, and X₅ are H;
Y is —(CH₂)—; and
Z is NH.

8. The method of claim 6, wherein
R₁ is —(CH₂)₅—;
W₁₋₄ are H;
X₁ and X₃ are NO₂;
X₂, X₄, and X₅ are H;
Y is —(CH₂)—; and
Z is NH.

9. A method of inhibiting ceramide glucosyltransferase and/or lowering a glycolipid concentration comprising administering to a subject in need thereof an effective amount of a compound of formula II or a pharmaceutically acceptable salt thereof: 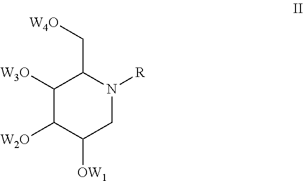
wherein R is: 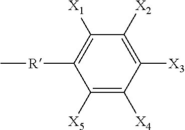
R′ is a substituted or unsubstituted alkyl group;
W₁₋₄ are independently selected from hydrogen, substituted or unsubstituted alkyl groups, substituted or unsubstituted haloalkyl groups, substituted or unsubstituted alkanoyl groups, substituted or unsubstituted aroyl groups, or substituted or unsubstituted haloalkanoyl groups; and
X₁₋₅ are independently selected from H, NO₂, halogen, alkyl, or halogenated alkyl.

10. The method of claim 9, wherein R′ is an unsubstituted or substituted alkyl group having from 1 to 12 carbon atoms.

11. The method of claim 10, wherein R′ is an alkyl group substituted with from 1 to 3 oxygen atoms.

12. The method of claim 11, wherein R′ is (CH2)ₙ—O—(CH₂)ₘ, where n is 5-8 and m is 0-4.

13. The method of claim 10, wherein R′ is an amino-substituted alkyl group.

14. The method of claim 13, wherein R′ is (CH₂)ₚ—NH—(CH₂)_{q}, where p is 5-8 and q is 0-2

15. The method of claim 9, wherein at least one of X₁₋₅ is halogen or halogenated alkyl.

16. The method of claim 9, wherein the compound of formula II has formula IIa:

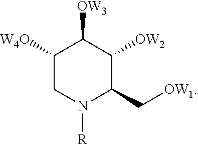

17. The method of claim 16, wherein R is selected from

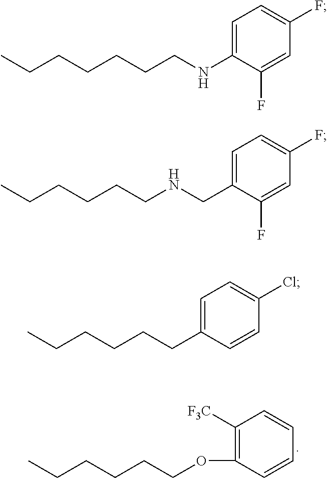

18. A method of inhibiting ceramide glucosyltransferase and/or lowering a glycolipid concentration comprising administering to a subject in need thereof an effective amount of a compound of formula 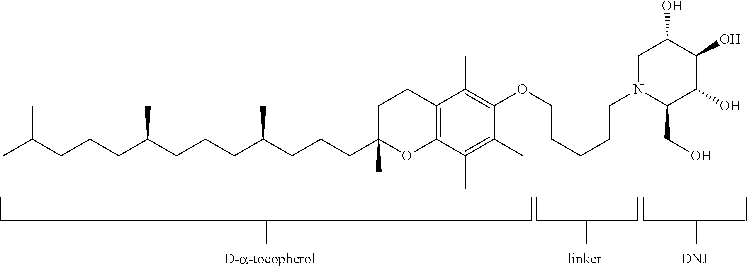 [SMILES: Cc1c(C)c2c(c(C)c1OCCCCCN1C[C@H](O)[C@@H](O)[C@H](O)[C@H]1CO)CC[C@@](C)(CCC[C@H](C)CCC[C@H](C)CCCC(C)C)O2]
or a pharmaceutically acceptable salt thereof.

19. The method of any one of claims 1-2, 9 and 18, wherein the subject is a subject with a disease or condition for which inhibiting ceramide glucosyltransferase and/or lowering a glycolipid concentration is beneficial, wherein said administering results in treatment of said disease or condition.

20. The method of claim 19, wherein the disease or condition is Gaucher disease, Fabry disease, Sandhoff disease, Tay-Sachs disease, GM1 Gangliosidosis, Niemann-Pick Type C disease, type 2 diabetes, hypertrophy or hyperplasia associated with diabetic nephropathy, an elevated blood glucose level, an elevated glycated hemoglobin level, a glomerular disease or lupus.

21. The method of claim 20, wherein the disease or condition is type I, type II or type III Gaucher disease.

22. The method of claim 21, wherein said administering results in chaperoning of β-glucocerebrosidase activity.

23. The method of claim 19, wherein the disease or condition is systemic lupus erythematous.

24. The method of any one of claims 1-2, 9 and 18, wherein the subject is a human being.

25. A method of inhibiting glycolipid biosynthesis in cells capable of producing glycolipids comprising subjecting said cells to a glycolipid inhibitory effective amount of N-(9-Methoxynonyl)deoxynojirimycin or a pharmaceutically acceptable salt thereof.

26. A method of inhibiting glycolipid biosynthesis in cells capable of producing glycolipids comprising subjecting said cells to a glycolipid inhibitory effective amount of a compound of Formula I or a pharmaceutically acceptable salt thereof: 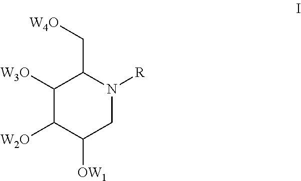
wherein R is: 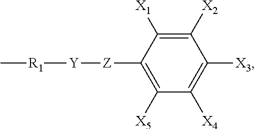
R₁ is a substituted or unsubstituted alkyl group;
W₁₋₄ are independently selected from hydrogen, substituted or unsubstituted alkyl groups, substituted or unsubstituted haloalkyl groups, substituted or unsubstituted alkanoyl groups, substituted or unsubstituted aroyl groups, or substituted or unsubstituted haloalkanoyl groups;
X₁₋₅ are independently selected from H, NO₂, N₃, or NH₂;
Y is absent or is a substituted or unsubstituted C₁-alkyl group, other than carbonyl; and
Z is selected from a bond or NH,
provided that when Z is a bond, Y is absent, and
provided that when Z is NH, Y is a substituted or substituted C₁-alkyl group, other than carbonyl.

27. A method of inhibiting glycolipid biosynthesis in cells capable of producing glycolipids comprising subjecting said cells to a glycolipid inhibitory effective amount of a compound of formula II or a pharmaceutically acceptable salt thereof: 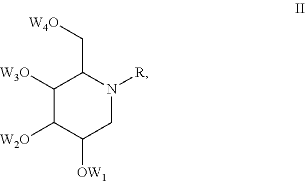
wherein R is: 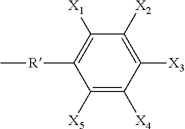
R′ is a substituted or unsubstituted alkyl group;
W₁₋₄ are independently selected from hydrogen, substituted or unsubstituted alkyl groups, substituted or unsubstituted haloalkyl groups, substituted or unsubstituted alkanoyl groups, substituted or unsubstituted aroyl groups, or substituted or unsubstituted haloalkanoyl groups; and
X₁₋₅ are independently selected from H, NO₂, halogen, alkyl, or halogenated alkyl.

28. The method of any one of claims 25-27, wherein said subjecting is performed in vitro.

29. The method of any one of claims 25-27, wherein the glycolipids comprise a glucoceramide based glycosphingolipid.

30. The method of any one of claims 25-27, wherein the glycolipids comprise GM3.

31. The method of any one of claims 25-27, wherein the cells are human cells.

32. A compound of formula I: 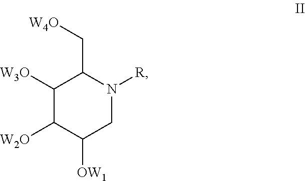
wherein R is: 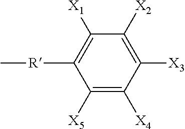
R′ is a substituted or unsubstituted alkyl group;
W₁₋₄ are independently selected from hydrogen, substituted or unsubstituted alkyl groups, substituted or unsubstituted haloalkyl groups, substituted or unsubstituted alkanoyl groups, substituted or unsubstituted aroyl groups, or substituted or unsubstituted haloalkanoyl groups; and
X₁₋₅ are independently selected from H, NO₂, halogen, alkyl, or halogenated alkyl.

33. The compound of claim 32, wherein R′ is an unsubstituted or substituted alkyl group having from 1 to 12 carbon atoms.

34. The compound of claim 32, wherein R′ is an alkyl group substituted with from 1 to 3 oxygen atoms.

35. The compound of claim 32, wherein R′ is (CH₂)ₙ—O—(CH₂)ₘ, where n is 5-8 and m is 0-4.

36. The compound of claim 32, wherein R′ is an amino-substituted alkyl group.

37. The compound of claim 32, wherein R′ is (CH2)p-NH—(CH₂)q, where p is 5-8 and q is 0-2.

38. The compound of claim 32, wherein at least one of X₁₋₅ ishalogen or halogenated alkyl.

39. The compound of claim 32, wherein at least one of X₃ and X₅ is halogen, NO₂ or halogenated alkyl and X₁, X₂ and X₄ are hydrogen.

40. The compound of claim 32, wherein at least one of X₃ and X₅ is F or Cl.

41. The compound of claim 32 having formula IIa:

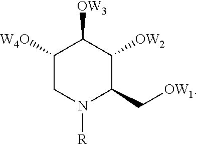

42. The compound of claim 32, wherein R is selected from [chemical structure]
